(19) United States
(12) Patent Application Publication
(10) Pub. No.: US 2017/0305871 A1
(43) Pub. Date: Oct. 26, 2017

(54) LONG-CHAINED MONO AND DI-ESTERS OF 2,5-DI(HYDROXYMETHYL)TETRAHYDROFURAN, USE AND PRODUCTION THEREOF

(71) Applicant: BASF SE, Ludwigshafen (DE)

(72) Inventors: Dominik Ohlmann, Mannheim (DE); Markus Dierker, Düsseldorf (DE); Vanessa Puetz, Elsdorf (DE); Stefan Busch, Düsseldorf (DE); Rene Backes, Lampertheim (DE); Anne Van Den Wittenboer, Düsseldorf (DE)

(73) Assignee: BASF SE, Ludwigshafen (DE)

(21) Appl. No.: 15/517,576

(22) PCT Filed: Aug. 12, 2015
(86) PCT No.: PCT/EP2015/068540

§ 371 Date: Apr. 7, 2017

(30) Foreign Application Priority Data
Oct. 7, 2014 (EP) 14187932.0

(51) Int. Cl. C07D 307/20; A61K 47/22; A61K 8/49; A61Q 19/00; C12P 17/04; C11D 1/66

(52) U.S. Cl. CPC C07D 307/20; C12Y 301/01003; C12P 17/04; C11D 1/667; A61K 8/4973; A61Q 19/00; A61K 47/22; A61K 2800/10

(57) ABSTRACT

The present invention relates to novel mono- and diesters of 2,5-di(hydroxymethyl)tetrahydrofuran, processes for their preparation by esterification in the presence of tertiary amines or in the presence of enzymatic esterification catalysts and the use of these mono- and diesters as interface-active compounds, rheology modifiers and emollients. The invention further relates to cosmetic and pharmaceutical compositions, and to detergents, cleaners and dishwashing compositions which comprise these mono- and diesters of 2,5-di(hydroxymethyl)-tetrahydrofuran.

### BACKGROUND OF THE INVENTION

The present invention relates to novel mono- and diesters of 2,5-di(hydroxymethyl)tetrahydrofuran, processes for their preparation by esterification in the presence of tertiary amines or in the presence of enzymatic esterification catalysts and the use of these mono- and diesters as interface-active compounds, rheology modifiers and emollients. The invention further relates to cosmetic and pharmaceutical compositions, and to detergents, cleaners and dishwashing compositions which comprise these mono- and diesters of 2,5-di(hydroxymethyl)tetrahydrofuran.

### PRIOR ART

Interface-active compounds (surfactant=surface active agent) have found extremely widespread use in industry. Depending on their chemical composition and application, interface-active substances are referred to as surfactants (detergents, soap), emulsifiers or wetting agents, in medicine (physiology) also as surfactant. As regards their primary effect, interface-active compounds specifically in the area of personal care and home care are also differentiated into surfactants, emulsifiers, solubilizers and foam formers.

Surfactants are generally understood as meaning interface-active compounds which lower the interfacial tension between two phases. They thus permit for example the better wetting of solid surfaces with a liquid by reducing the surface tension or the formation of dispersions. They are used in very diverse areas, e.g. as washing- and cleaning-active substances in detergents, dishwashing detergents, cleaners and compositions for body care, and also as solubilizers or wetting agents, e.g. in cosmetics, pharmacy, the food industry, material protection, crop protection, etc.

Emulsifiers is the term generally used to refer to interface-active compounds which serve to mix two immiscible liquids, such as for example water and an oil body, to give an emulsion and to stabilize this against separation.

Solubilizers increase the solubility of a substance in a solvent as a result of the addition of a third substance. Here, two effects are differentiate: as solubility promoters, which change the dissolution properties of the solvent by homogeneous mixing, and as surfactants, which increase the solubility as a result of micelle formation.

Emollients are caring and hydrating skincare agents (e.g. in the form of ointments, creams and lotions) which supply the skin with moisture and fats. They are used for a large number of skin conditions and are in most cases free from pharmaceutical active ingredients. Some comprise moisture-donating or -binding substances, such as for example urea or lactic acid.

The rheology describes a force which acts on a material and which leads to liquids flowing in order to deflect the pressure of this force. All materials counteract an applied shear force. This resistance is an internal force which generally proceeds tangentially to the surface under stress. The measurement of this resistance gives the viscosity, a fundamental value of the rheology. Rheological behavior knows two extremes, firstly elastic behavior (absolutely rigid solids): the effect of an applied force changes spontaneously to its opposite if it is lifted. The system stores energy and releases it again as a result. Secondly, viscous or plastic behavior (ideal Newtonian liquids): a deformation immediately diminishes if the acting force is removed.

The rheology of flowable systems is of great importance for many areas of technology. In order to adjust the viscosity behavior of fluid components to the application-specific requirements in each case, such as e.g. the provision of a higher-viscosity liquid composition, of a paste or of a gel, rheological additives (so-called rheology modifiers) can be incorporated into existing flowable systems. Rheology modifiers in the context of the invention serve to influence the viscosity of flowable compositions in the desired manner, and also as consistency regulators, structure-imparting agents, gel formers, etc.

A large number of requirements that have to be satisfied simultaneously is applied specifically to products from the sectors homecare, personal care, pharmacy and agrochemistry, but also in many other areas, both as regards their effectiveness and also the confection and supply forms. Good application properties should be accompanied with convenient dosing by the consumer and simplicity of the operating steps necessary to carry out a treatment process. In many cases, it is precisely end consumers who also place requirements on optical and haptic properties. Thus, e.g. in the sector of cleaning skin and hair and in the case of detergents, cleaners and dishwashing compositions, preference is given to products which develop a good cleaning performance and which are nevertheless flowable, but not too readily flowable or viscous, in order to permit optimum application. For skin cleansers, it is often desirable that they supply the skin with moisture or have a refatting effect. There is also a great need here both for effect-active substances and also for components which positively influence the rheology. Preference is given here to components which develop a multiple effect, e.g. as rheology-modifying component which is soluble under the application conditions and, after its dissolution, is involved in the cleaning process. Furthermore, there is a need for components which originate at least partly from biogenic sources and can be produced specifically from renewable materials.

It is known to use quite generally fatty acid esters in cosmetic and pharmaceutical products in which they are used inter alia as oil bodies and emollients. It is likewise known to prepare esters from fatty alcohols and fatty acids by enzymatic synthesis. The industrial application of lipases for producing fatty acid esters, such as decyl oleate, cetyl ricinoleate, myristyl myristate or decyl cocoate, has been described for example by Geoffrey Hills in Eur. J. Lipid Sci. Technol. 105, 2003, pages 601-607.

U.S. Pat. No. 4,826,767 describes the enzymatic synthesis of esters of fatty acids and fatty alcohols in liquid phase and in vacuo in the presence of an immobilized lipase.

EP 2 080 807 A2 describes a process for the enzymatic preparation of carboxylic acid esters, where the mixing of the reactants and the discharge of the water of reaction take place by introducing a gas.

WO 2014/056756 describes a three-stage process for enzymatic fatty acid synthesis in which fatty alcohols and fatty acids are reacted in the presence of an enzyme at a temperature in the range from 30 to 50° C., the water formed is removed and then the reaction is completed in vacuo at a temperature of 50 to 80° C.

Enzymatic ester syntheses using 2,5-di(hydroxymethyl)tetrahydrofuran or structurally related diols are not known.

2,5-Di(hydroxymethyl)tetrahydrofuran (IUPAC: [5-(hydroxymethyl)oxolan-2-yl]methanol, also called THF-Glycol) is commercially available and is used for example for producing plasticizers, polymer resins and as solvent.

A number of short-chain mono- and diesters (A) of 2,5-di(hydroxymethyl)tetrahydrofuran are also already known.

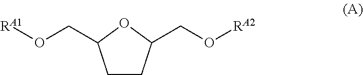

Monoesters: R^{A1}=acyl, R^{A2}=H, diesters: R^{A1}=acyl, R^{A2}=acyl

Jung et al. describe in Heterocycles 1993, 35, 273-280 the synthesis of diesters of 2,5-di(hydroxymethyl)tetrahydrofuran, where the diol is reacted with Mosher's acid (α-methoxy α-trifluoromethylphenylacetic acid) in the presence of dicyclohexylcarbodiimide and 4-dimethylaminopyridine. The discrimination of the diastereomeric diols thus takes place into those with C_{S} symmetry and those with C₂ symmetry.

C. E. Müller et al. describe in J. Org. Chem. 2013, 78, 8465-8484 the use of lipophilic oligopeptides as organocatalysts for chemo- and enantioselective acylations. The monoacetylation of 2,5-di(hydroxymethyl)tetrahydrofuran to 2-(acetoxymethyl)-5-(hydroxymethyl)tetrahydrofuran is possible by this method.

H. Estermann, K. Prasad and M. J. Shapiro describe in Tetrahedron Lett. 1990, Vol. 31, No. 4, 445-448 the enzymatic partial saponification of 2,5-di(butyryloxymethyl)-tetrahydrofuran to 2-(butyryloxymethyl)-5-(hydroxymethyl)tetrahydrofuran.

K. Naemura et al. describe in Tetrahedron Asymmetry 1993, 4, 911-918 the enzyme-catalyzed asymmetric acylation and hydrolysis of cis-2,5-disubstituted tetrahydrofuran derivatives. The compounds (B), (C) and (D), inter alia, could be obtained in the process.

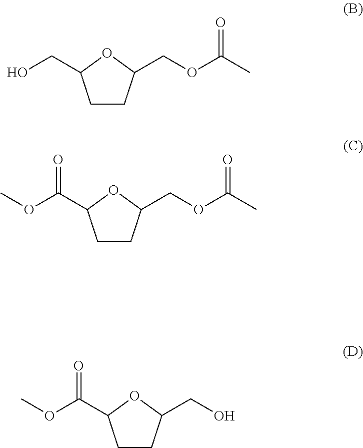

They are intended to serve as synthesis building blocks for producing new antibiotics.

The previously unpublished international application PCT/EP2014/057411 describes tetrahydrofuran derivatives of the general formula (E),

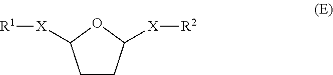

in which
X is *—(C═O)—O—, *—(CH₂)ₙ—O— or *—(CH₂)ₙ—O—(C═O)—, where * is the linkage point with the tetrahydrofuran ring and n has the value 0, 1 or 2, and
R¹ and R², independently of one another, are selected from C₄-C₅-alkyl and C₅-C₆-cycloalkyl, where the cycloalkyl radicals are unsubstituted or can be substituted by at least one C₁-C₁₀-alkyl radical.

They serve as plasticizers for thermoplastic polymers, in particular polyvinyl chloride (PVC). An embodiment of a diester of 2,5-di(hydroxymethyl)tetrahydrofuran is not present in this application.

The previously unpublished European patent application 13182979.8 describes tetrahydrofuran derivatives of the general formula (F),

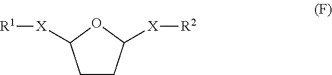

in which
X is *—(C═O)—O—, *—(CH₂)ₙ—O— or *—(CH₂)ₙ—O—(C═O)—, where * is the linkage point with the tetrahydrofuran ring and n has the value 0, 1 or 2;
R¹ and R², independently of one another, are selected from unbranched and branched C₇-C₁₂-alkyl radicals.

They likewise serve as plasticizers for thermoplastic polymers, in particular polyvinyl chloride (PVC). The only diester of 2,5-di(hydroxymethyl)tetrahydrofuran demonstrated by an example is 2,5-di(hydroxymethyl)tetrahydrofuran diethyl hexanoate.

The object of the present invention is to provide compounds which are advantageously suitable for use as interface-active compounds, rheology modifiers or emollients for diverse applications. They should specifically be suitable for covering a complex spectrum of requirements as described at the outset.

Surprisingly, it has now been found that this object is achieved through the use of tetrahydrofuran derivatives which carry, in the 2 position and optionally additionally in the 5 position, an acyclic or cyclic hydrocarbon radical bonded via an ester group or an ether group.

### SUMMARY OF THE INVENTION

A first subject matter of the invention is compounds of the general formula (I)

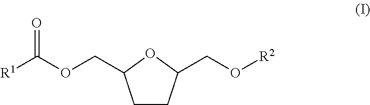

in which
R² is C(═O)R^{1′} and where R¹ and R^{1′}, independently of one another, are unbranched or branched C₈-C₃₅-alkyl or unbranched or branched C₈-C₃₅-alkenyl with 1, 2, 3 or more than 3 double bonds, where C₈-C₃₅-alkyl and C₈-C₃₅-alkenyl can in each case be substituted by at least one hydroxyl group,
or
R² is hydrogen and R¹ is unbranched or branched C₅-C₃₅-alkyl or unbranched or branched C₅-C₃₅-alkenyl with 1, 2, 3 or more than 3 double bonds, where C₅-C₃₅-alkyl and C₅-C₃₅-alkenyl can be substituted by at least one hydroxyl group and/or can have at least one epoxy group.

In a preferred embodiment, at least one of the radicals R¹ and R^{1′} is unbranched or branched C₁₁-C₂₁-alkyl or unbranched or branched C₁₁-C₂₁-alkenyl with 1, 2 or 3 double bonds, where C₁₁-C₂₁-alkyl and C₁₁-C₂₁-alkenyl can be substituted by at least one hydroxyl group.

A further subject matter of the invention is a process for the preparation of compounds of the general formula (I), as defined above and below, in which 2,5-di(hydroxymethyl)tetrahydrofuran is reacted with at least one compound R¹—COOH and, if R² is —(C═O)R^{1′} and R¹ and R^{1′} have a different meaning, additionally with a compound R^{1′}—COOH different therefrom in the presence of an enzymatic esterification catalyst.

A further subject matter of the invention is a process for the preparation of compounds of the general formula (I), as defined above and below, in which 2,5-di(hydroxymethyl)tetrahydrofuran is reacted with at least one acid halide R¹—C(═O)X and, if R² is C(═O)R^{1′} and R¹ and R^{1′} have a different meaning, additionally with at least one acid halide R¹¹—C(═O)X, where X is Br or Cl, in the presence of at least one tertiary amine.

A further subject matter of the invention is a cosmetic or pharmaceutical composition comprising at least one compound of the general formula (I), as defined above and below, and at least one cosmetic or pharmaceutical active ingredient and/or auxiliary different therefrom.

A further subject matter of the invention is a detergent, cleaner or dishwashing composition comprising at least one compound of the general formula (I), as defined above and below, and at least one surfactant different therefrom.

A further subject matter of the invention is the use of at least one compound of the general formula (I), as defined above and below, as interface-active compound. In this connection, the compounds of the general formula (I) are advantageously suitable as surfactants, emulsifiers, solubilizers and foam formers. Compounds of the general formula (I) preferred and suitable for the individual uses are described in more detail below.

A further subject matter of the invention is the use of at least one compound of the general formula (I), as defined above and below, as rheology modifier. Compounds of the general formula (I) preferred and suitable for use as rheology modifiers are likewise described in more detail below.

A further subject matter of the invention is the use of at least one compound of the general formula (I), as defined above and below, as emollients. Compounds of the general formula (I) preferred and suitable for use as emollients are likewise described in more detail below.

The compounds of the general formula (I), as defined above and below, are suitable in an advantageous manner for use
in cosmetic compositions,
in pharmaceutical compositions,
in detergents and cleaners,
in dishwashing compositions and in rinse aids,
in hygiene products,
in crop protection compositions,
in paints, coating compositions, adhesives, leather-treatment or textile-treatment compositions, etc.,
in the development and/or exploitation of subterranean natural oil and/or natural gas deposits.

### DESCRIPTION OF THE INVENTION

Unless stated more precisely below, the terms

2-(acyloxymethyl)-5-(hydroxymethyl)tetrahydrofuran and 2,5-di(acyloxymethyl)tetrahydrofuran
in the context of the invention refer to cis/trans mixtures of any composition and also to the pure configurational isomers. The aforementioned terms furthermore relate to all enantiomers in pure form and also to racemic and optically active mixtures of the enantiomers of these compounds.

Wherever cis and transdiastereomers of the compounds (I) are discussed hereinbelow, in each case only one of the enantiomeric forms is depicted. Merely for the purposes of illustration, the isomers of 2-(acyloxymethyl)-5-(hydroxymethyl)tetrahydrofuran are given below:

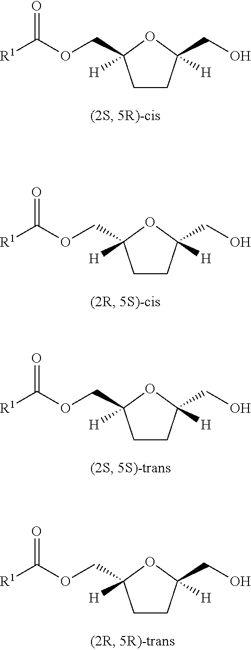

The terms “rheology modifier” and “modification of rheological properties” in the context of the present invention are understood in the wide sense. The correspondingly used compounds of the formula (I) are suitable in general for thickening the consistency of liquid compositions in a wide range. Depending on the basic consistency of the liquid compositions, flow properties from thin-liquid ranging to solid (in the sense of “no longer flowable”) can be generally achieved depending on the use amount of the compounds of the general formula (I). “Modification of rheological properties” is therefore understood as meaning inter alia the increase in the viscosity of liquids, the improvement in the thixotropy properties of liquids and gels, the solidification of gels and waxes etc. The compounds of the formula (I) are specifically suitable for modifying the rheological properties of aqueous compositions.

The term “solubilization” in the context of the present invention is also understood broadly in the sense of a solubility improvement. This includes firstly the stabilization of heterogeneous systems in which the sparingly soluble substance is present as emulsified or dispersed phase (disperse phase) in a liquid (e.g. aqueous) medium as continuous phase. This also includes the stabilization of transition stages to homogeneous solutions, such as colloidal solutions, etc., ranging to molecularly disperse solutions. It also includes a solubility improvement in the sense of a solubilization in which the poorly soluble or insoluble substances are converted to solutions, which are preferably clear or at most opalescent. Finally, it also includes the ability to form so-called “solid solutions”.

A low (poor) solubility in the context of this invention means a solubility in a solvent (specifically in water) of less than 10 g/l, in particular less than 1 g/l and specifically less than 0.1 g/l at 25° C. and 1013 mbar.

Suitable C₅-C₃₅-alkyl groups, C₈-C₃₅-alkyl groups, C₁₁-C₃₅-alkyl groups, C₁₁-C₂₁-alkyl groups, C₅-C₂₃-alkyl groups, C₅-C₃₅-alkenyl groups, C₈-C₃₅-alkenyl groups, C₁₁-C₃₅-alkenyl groups, C₁₁-C₂₁-alkenyl groups and C₅-C₂₃-alkyl groups are in each case straight-chain and branched alkyl or alkenyl groups.

Preferably, they are predominantly linear alkyl radicals, as also occur in natural or synthetic fatty acids and fatty alcohols and also oxo alcohols, or are predominantly linear alkenyl radicals, as also occur in natural or synthetic fatty acids and fatty alcohols and also oxo alcohols, which can be mono-, di-, tri-, tetra-, penta- or hexaunsaturated. If the alkenyl radical comprises more than one carbon-carbon double bond, these are preferably not vicinal, i.e. not allenic.

If the radical R¹ or if the radicals R¹ and R^{1′} are alkyl radicals or alkenyl radicals, then these preferably originate from natural raw materials, particularly preferably from a renewable raw material.

If the radical R¹ or if the radicals R¹ and R^{1′} are an unbranched or branched C₁₁-C₂₁-alkyl, then these are preferably selected from n-undecyl, n-dodecyl, n-tridecyl, n-tetradecyl, n-pentadecyl, n-hexadecyl, n-heptadecyl, n-octadecyl, n-nonadecyl, arachinyl, isotridecyl, isostearyl, and constitutional isomers thereof.

If the radical R¹ or if the radicals R¹ and R^{1′} are unbranched or branched C₁₁-C₃₅-alkyl, then these are preferably selected from the C₁₁-C₂₁-alkyl radicals listed above, behenyl, lignocerinyl, cerotinyl, melissinyl, and constitutional isomers thereof.

If the radical R¹ or if the radicals R¹ and R^{1′} are unbranched or branched C₈-C₃₅-alkyl, then these are preferably selected from the C₁₁-C₃₅-alkyl radicals listed above, n-octyl, 2-ethylhexyl, n-nonyl, n-decyl, and constitutional isomers thereof.

If the radical R¹ or if the radicals R¹ and R^{1′} are unbranched or branched C₅-C₃₅-alkyl, then these are preferably selected from the C₈-C₃₅-alkyl radicals listed above, n-pentyl, n-hexyl, n-heptyl, and constitutional isomers thereof.

If the radical R¹ or if the radicals R¹ and R^{1′} are unbranched or branched C₅-C₃₅-alkyl, C₈-C₃₅-alkyl, C₁₁-C₃₅-alkyl or C₁₁-C₂₁-alkyl which is substituted by at least one hydroxyl group, then they are preferably derived from the aforementioned C₅-C₃₅-alkyl, C₈-C₃₅-alkyl, C₁₁-C₃₅-alkyl or C₁₁-C₂₁-alkyl groups which carry at least one (e.g. 1, 2, 3, 4 or more than 4) hydroxyl groups. Examples are 6-hydroxyhexyl, 7-hydroxyheptyl, 8-hydroxyoctyl, 9-hydroxynonyl, 10-hydroxydecyl, 11-hydroxyheptadecyl, 8,9-bis-hydroxyheptadecyl and 3-hydroxyheptadecyl.

In a preferred embodiment, the radical R¹ or the radicals R¹ and R^{1′} is/are unbranched or branched C₅-C₃₅-alkyl which is substituted by 1, 2 or 3 hydroxyl groups.

In a further preferred embodiment, the radical R¹ or the radicals R¹ and R^{1′} is/are unbranched or branched C₈-C₃₅-alkyl which is substituted by 1, 2 or 3 hydroxyl groups.

In a further preferred embodiment, the radical R¹ or the radicals R¹ and R^{1′} are unbranched or branched C₁₁-C₂₁-alkyl which is substituted by 1, 2 or 3 hydroxyl groups.

If the radical R¹ is unbranched or branched C₅-C₃₅-alkyl, C₈-C₃₅-alkyl, C₁₁-C₃₅-alkyl or C₁₁-C₂₁-alkyl, has at least one epoxy group, then they are preferably derived from the aforementioned C₅-C₃₅-alkyl, C₈-C₃₅-alkyl, C₁₁-C₃₅-alkyl- or C₁₁-C₂₁-alkyl groups which carry at least one (e.g. 1, 2, 3, 4 or more than 4) epoxy groups.

In a preferred embodiment, the radical R¹ is unbranched or branched C₅-C₃₅-alkyl which carries 1, 2, 3 or 4 epoxy groups.

In a further preferred embodiment, the radical R¹ is unbranched or branched C₈-C₃₅-alkyl which carries 1, 2, 3 or 4 epoxy groups.

In a further preferred embodiment, the radical R¹ is unbranched or branched C₁₁-C₂₁-alkyl which carries 1, 2, 3 or 4 epoxy groups.

Suitable C₅-C₃₅-alkenyl groups, C₈-C₃₅-alkenyl groups, C₁₁-C₃₅-alkenyl groups and C₁₁-C₂₁-alkenyl groups are in each case straight-chain and branched alkenyl groups which can be mono-, di-, tri- or more than triunsaturated.

If the radical R¹ or if the radicals R¹ and R^{1′} are unbranched or branched C₁₁-C₂₁-alkenyl, then they are preferably selected from n-undecenyl, n-dodecenyl, n-tridecenyl, n-tetradecenyl, n-pentadecenyl, n-hexadecenyl, n-heptadecenyl, n-octadecenyl, n-nonadecenyl, n-eicosenyl, linolenyl, eleostearyl and oleyl (9-octadecenyl) and constitutional isomers thereof.

If the radical R¹ or if the radicals R¹ and R^{1′} are unbranched or branched C₁₁-C₃₅-alkenyl, then they are preferably selected from the C₁₁-C₂₁-alkenyl radicals listed above, n-docosenyl, n-tetracosenyl, n-hexacosenyl, and constitutional isomers thereof.

If the radical R¹ or if the radicals R¹ and R^{1′} are unbranched or branched C₈-C₃₅-alkenyl, then they are preferably selected from the C₁₁-C₃₅-alkenyl radicals listed above, n-octenyl, n-nonenyl, n-decenyl, and constitutional isomers thereof.

If the radical R¹ or if the radicals R¹ and R^{1′} are unbranched or branched C₅-C₃₅-alkenyl, then they are preferably selected from the C₈-C₃₅-alkenyl radicals listed above, n-pentenyl, n-hexenyl, n-heptenyl, and constitutional isomers thereof.

The radicals R¹ and R^{1′}, which are C₅-C₃₅-alkyl, C₈-C₃₅-alkyl, C₁₁-C₃₅-alkyl, C₁₁-C₂₁-alkyl, C₅-C₃₅-alkenyl, C₈-C₃₅-alkenyl, C₁₁-C₃₅-alkenyl and C₁₁-C₂₁-alkenyl, can be derived from the corresponding carboxylic acids as a result of formal cleaving off of the COOH group. The radicals R¹ and R² can be derived from pure carboxylic acids or from carboxylic acid mixtures. These are preferably industrially available carboxylic acids or carboxylic acid mixtures. In one preferred embodiment, R¹ and R^{1′} are then independently of one another selected from predominantly linear alkyl and alkenyl, alkadienyl radicals, as occur in natural or synthetic fatty acids. In one suitable embodiment, R¹ and R² are independently of one another derived from fatty acids which are based on technical fatty acid mixtures. Preferably, R¹ and R^{1′} are independently of one another derived from naturally occurring fatty acids and fatty acid mixtures.

Naturally occurring fatty acids and fatty acid mixtures are preferred as monocarboxylic acid. These are present in nature as oils or fats in the form of triglycerides. They can be used for the preparation of the compounds (I) according to the invention in the form of the free fatty acid or a derivative, specifically in the form of an acid halide, ester or anhydride.

Suitable saturated aliphatic monocarboxylic acids are, for example, caproic acid, enanthic acid, caprylic acid, perlargonic acid, capric acid, undecanoic acid, lauric acid, tridecanoic acid, myristic acid, pentadecanoic acid, palmitic acid, margaric acid, stearic acid, nonadecanoic acid, tuberculostearic acid, arachic acid, behenic acid, etc.

Suitable unsaturated monocarboxylic acids have at least one double bond (unsaturation). They can also have 2, 3, 4, 5 or 6 double bonds. The double bonds can in each case have (E)- or (Z)-configuration.

Preferred monounsaturated carboxylic acids are undecenoic acid (C11), dodecenoic acid (C12), tridecenoic acid (C13), tetradecenoic acid (C14), pentadecenoic acid (C15), hexadecenoic acid (C16), heptadecenoic acid (C17), octadecenoic acid (C18), nonadecenoic acid (C19), eicosenoic acid (C20), docosenoic acid (C22), tetracosenoic acid (C24). Here, the unsaturation can occur at any position in the alkenyl chain. The double bonds can in each case have (E)- or (Z)-configuration.

In a preferred embodiment of the invention, linear, monounsaturated carboxylic acids are used. These are preferably selected from n-undecenoic acid (C11), n-dodecenoic acid (C12), n-tridecenoic acid (C13), n-tetradecenoic acid (C14), n-pentadecenoic acid (C15), n-hexadecenoic acid (C16), n-heptadecenoic acid (C17), n-octadecenoic acid (C18), n-nonadecenoic acid (C19), n-eicosenoic acid (C20), n-docosenoic acid (C22), n-tetracosenoic acid (C24). Here, the unsaturation can arise at any position in the alkenyl chain. The double bonds can in each case have (E)- or (Z)-configuration.

Preferred examples of monounsaturated linear carboxylic acids are:

(Z)-undec-10-enoic acid
(E)-undec-10-enoic acid
myristoleic acid (IUPAC: (Z)-tetradec-9-enoic acid, C14:1, [omega]-5)
palmitoleic acid (IUPAC: (Z)-hexadec-9-enoic acid; 16:1, [omega]-7)
oleic acid (IUPAC: (Z)-octadec-9-enoic acid; 18:1 [omega]-9)
elaic acid (IUPAC: (E)-octadec-9-enoic acid; C18:1, [omega]-9)
erucic acid (IUPAC (Z)-docos-13-enoic acid; 22:1 [omega]-9)
(Z)-octadec-2-enoic acid (18:1)
nervonic acid (IUPAC: (Z)-tetracos-15-enoic acid; C24:1, [omega]-9)
vaccenic acid, [(E)-octadec-11-enoic acid; C18:1],
petroselic acid [(Z)-6-octadecenoic acid] C18:1.

Examples of diunsaturated linear carboxylic acids are:

Linolenic acid (IUPAC (all-Z)-9,12-octadecadienoic acid; 18:2 [omega]-6)
(all-E)-9,12-octadecadienoic acid (18:2 [omega]-6).

Examples of triunsaturated linear carboxylic acids are:

Alpha-linolenic acid ((all-Z)-9,12,15-octadecatrienoic acid; C18:3 [omega]-3)
gamma-linolenic acid ((all-Z)-6,9,12-octadecatrienoic acid; GLA; C18:3, [omega]-6)
eleostearic acids, (octadeca-9,11,13-trienoic acid; C18:3), such as alpha-eleostearic acid [(9Z,11E,13E)-9,11,13-octadeca-9,11,13-trienoic acid]
di-homo-gamma-linolenic acid ((all-Z)-8,11,14-eicosatrienoic acid, C20:3).

Examples of tetraunsaturated linear carboxylic acids are:

Arachidonic acid (IUPAC: (all-Z)-5,8,11,14-eicosatetraenoic acid, C20:4, [omega]-6)
stearidonic acid (IUPAC: (all-Z)-6,9,12,15-octatetraenoic acid, C18:4, [omega]-3).

Examples of pentaunsaturated linear carboxylic acids are:

Clupa(no)donic acid [(all-Z)-4,8,12,15,19-docosapentaenoic acid, C22:5]
eicosapentaenoic acid [(all-Z)-5,8,11,14,17-icosapentaenoic acid, EPA, C22:5].

Examples of hexaunsaturated linear carboxylic acids are:

Docosahexaenoic acid [(4Z,7Z,10Z,13Z,16Z,19Z)-docosa-4,7,10,13,16,19-hexaenoic acid, C22:6, cervonic acid, [omega]-3].

In the compounds of the general formula (I), preferably at least one of the radicals R¹ and R^{1′} is unbranched or branched C₁₁-C₂₁-alkyl or unbranched or branched C₁₁-C₂₁-alkenyl with 1, 2 or 3 double bonds, where C₁₁-C₂₁-alkyl and C₁₁-C₂₁-alkenyl can be substituted by at least one hydroxyl group.

In a preferred embodiment, in the compounds of the general formula (I), the radical R¹ or the radicals R¹ and R^{1′} is/are unsubstituted unbranched or branched C₁₁-C₂₁-alkyl.

In a further preferred embodiment, in the compounds of the general formula (I), the radical R¹ or the radicals R¹ and R^{1′} is/are unbranched or branched C₁₁-C₂₁-alkyl which is substituted by 1, 2 or 3 hydroxyl groups.

In a preferred embodiment, in the compounds of the general formula (I), the radical R¹ or the radicals R¹ and R^{1′} is/are unsubstituted unbranched or branched C₁₁-C₂₁-alkenyl with 1, 2 or 3 double bonds.

In a further preferred embodiment, in the compounds of the general formula (I), the radical R¹ or the radicals R¹ and R^{1′} is/are unbranched or branched C₁₁-C₂₁-alkenyl with 1, 2 or 3 double bonds which is substituted by 1, 2 or 3 hydroxyl groups.

In a further preferred embodiment, in the compounds of the general formula (I), the radical R¹ or the radicals R¹ and R^{1′} is/are unbranched or branched C₁₁-C₂₁-alkenyl with 1, 2 or 3 double bonds which carries 1, 2, 3 or 4 epoxy groups.

In a first preferred variant, in the compounds of the general formula (I) R² is C(O)R^{1′} and the radicals R¹ and R^{1′} have the same meaning.

In a further preferred variant, in the compounds of the general formula (I), R² is hydrogen.

Furthermore, the radicals R¹ and R^{1′} are particularly preferably n-dodecyl (lauryl), n-tridecyl, n-tetradecyl (myristyl), n-pentadecyl, n-hexadecyl (palmitinyl), n-heptadecyl (margarinyl) or n-octadecyl (stearyl).

2,5-Di(hydroxymethyl)tetrahydrofuran is obtainable for example by hydrogenation of 2,5-di(hydroxymethyl)furan. 2,5-Di(hydroxymethyl)furan can be prepared e.g. starting from fructose by dehydrogenation to 5-hydroxymethylfurfural and subsequent reduction of the formyl group. Consequently, the preparation of 2,5-di(hydroxymethyl)tetrahydrofuran from biogenic sources, starting from corresponding carbohydrates, e.g. starch, cellulose and sugars, is possible.

Alternatively, the preparation of the mono- and diesters according to the invention of 2,5-di(hydroxymethyl)tetrahydrofuran can also take place via the corresponding mono- and diesters of 2,5-di(hydroxymethyl)furan, and these are ultimately subjected to a hydrogenation.

In a specific embodiment, the starting materials used for the preparation of the compounds of the general formula (I) originate at least partially from a renewable source, or their preparation takes place from renewable raw materials. In the context of the invention, renewable sources are understood as meaning natural (biogenic) sources and nonfossil sources, such as natural oil, natural gas or coal. Compounds obtained from renewable sources have a different ¹⁴C-to-¹²C-isotope ratio than compounds obtained from fossil sources, such as natural oil. The compounds of the general formula (I) therefore preferably have a ¹⁴C-to-¹²C-isotope ratio in the range from 0.5×10⁻¹² to 5×10-12.

### Preparation of the Compounds of the General Formula (I)

A further subject of the invention is a process for the preparation of compounds of the general formula (I)

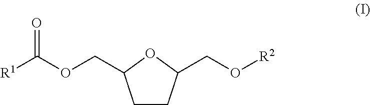

in which
R² is C(═O)R^{1′} and where R¹ and R^{1′}, independently of one another, are unbranched or branched C₈-C₃₅-alkyl or unbranched or branched C₈-C₃₅-alkenyl with 1, 2, 3 or more than 3 double bonds, where C₈-C₃₅-alkyl and C₈-C₃₅-alkenyl can in each case be substituted by at least one hydroxyl group,
or
R² is hydrogen and R¹ is unbranched or branched C₅-C₃₅-alkyl or unbranched or branched C₅-C₃₅-alkenyl with 1, 2, 3 or more than 3 double bonds, where C₅-C₃₅-alkyl and C₅-C₃₅-alkenyl can be substituted by at least one hydroxyl group and/or can have at least one epoxy group,
in which
2,5-di(hydroxymethyl)tetrahydrofuran is reacted with at least one acid halide R¹—C(═O)X and, if R² is —(C═O)R^{1′}, and R¹ and R^{1′} have a different meaning, additionally with at least one acid halide R^{1′}—C(═O)X, where X is Br or Cl, in the presence of at least one tertiary amine.

For the purposes of the esterification, all types of tertiary amines known to the person skilled in the art can be used. Examples of suitable tertiary amines are:
from the group of trialkylamines: trimethylamine, triethylamine, tri-n-propylamine, diethylisopropylamine, diisopropylethylamine and the like;
from the group of N-cycloalkyl-N,N-dialkylamines: dimethylcyclohexylamine and diethylcyclohexylamine;
from the group of N,N-dialkylanilines: dimethylaniline and diethylaniline;
from the group of pyridine and quinoline bases: pyridine, α-, β- and γ-picoline, quinoline and 4-(dimethylamino)pyridine (DMAP).

Preferred tertiary amines are trialkylamines and pyridine bases, in particular triethylamine and 4-(dimethylamino)pyridine (DMAP), and mixtures thereof.

In a preferred embodiment, a trialkylamine, preferably selected from trimethylamine, triethylamine, tri-n-propylamine, diethylisopropylamine and diisopropylethylamine, is used for the esterification. The trialkylamine here is preferably used in an at least stoichiometric ratio based on the hydroxyl groups of the 2,5-di(hydroxymethyl)-tetrahydrofuran. Particularly preferably, the trialkylamine is used in an at least stoichiometric ratio ranging to a four-fold stoichiometric excess, based on the hydroxyl groups of the 2,5-di(hydroxymethyl)tetrahydrofuran.

Preferably, dimethylaminopyridine is also used for the esterification in addition to at least one trialkylamine. It is assumed that dimethylaminopyridine acts here as a catalyst. The use amount of the dimethylaminopyridine is preferably in a range from 0.01 to 0.5 mole equivalents, particularly preferably 0.05 to 0.2 mole equivalents, based on 2,5-di(hydroxymethyl)tetrahydrofuran.

Preferably, the esterification is carried out in a temperature range from −10 to 75° C., preferably 0 to 60° C. At these low temperatures, a thermal decomposition of the 2,5-di(hydroxymethyl)tetrahydrofuran is avoided, as is observed for the processes known from the prior art in a strongly acidic medium and at elevated temperature. Furthermore, at these low temperatures, a good selectivity is achieved with regard to the respective process product (monoester or diester).

The esterification can take place at ambient pressure, at reduced pressure or increased pressure. Preferably, the esterification is carried out at ambient pressure.

The esterification can be carried out in the absence or presence of an organic solvent. Preferably, the reaction is carried out in the presence of an inert organic solvent, preferably selected from aliphatic ethers, cyclic ethers, ketones, chlorinated hydrocarbons, aliphatic hydrocarbons, cycloaliphatic hydrocarbons, aromatics and mixtures thereof. Suitable solvents are THF, dioxane, methyl tert-butyl ether, diethyl ether, acetone, methyl ethyl ketone, dichloromethane, trichloromethane, tetrachloromethane, pentane, hexane, heptane, ligroin, petroleum ether, cyclohexane, benzene, toluene, xylene, chlorobenzene, dichlorobenzenes and mixtures thereof. Preferably, the reaction is carried out in the presence of an essentially anhydrous organic solvent.

The alkylation can take place in the absence or presence of a gas inert under the reaction conditions, such as nitrogen or argon. Preferably, no inert gas is used for the esterification.

The ratio of the resulting monoesters to the diesters can be controlled via the stoichiometry of the reactants.

For the preparation of the pure diesters, the acid halide R¹—(C═O)X or the total amount of the acid halides R¹—(C═O)X and R^{1′}—(C═O)X is used in an at least equimolar amount or a molar excess, based on the hydroxyl groups of the 2,5-di(hydroxymethyl)-tetrahydrofuran. For the preparation of monoester-containing mixtures, the acid halide R¹—(C═O)X or the total amount of the acid halides R¹—(C═O)X and R^{1′}—(C═O)X is used in a corresponding deficit, based on the hydroxyl groups of the 2,5-di(hydroxymethyl)tetrahydrofuran.

A further subject of the invention is a process for the preparation of compounds of the general formula (I), as defined above and below, in which 2,5-di(hydroxymethyl)-tetrahydrofuran is reacted with at least one compound R¹—COOH and, if R² is C(═O)R^{1′} and R¹ and R^{1′} have a different meaning, additionally with a compound R¹¹—COOH different therefrom in the presence of an enzymatic esterification catalyst.

Preferably, the enzymatic esterification catalyst comprises at least one enzyme which is selected from hydrolases, preferably lipases, or esterases. Particularly preferably, the enzyme is selected from lipases from Candida rugosa, Candida antarctica, Thermomyces lanunginosa, Rhizomucor miehei or esterase from pig liver. Particular preference is given to lipases, specifically lipases from Candida antarctica, very specifically lipase B from Candida antarctica.

The enzymatic esterification catalyst can be used as cell extract, purified protein solution (homogeneous catalyst) or in immobilized (bonded to a support) form (heterogeneous catalyst). Preferably, the enzyme is used in immobilized form.

For the immobilization, the enzymes are preferably bonded either adsorptively, ionically or covalently to inorganic or organic support particles. This belongs to the knowledge of the appropriate person skilled in the art.

Organic supports that can be used are in particular those which have polyacrylate, polymethacrylate, polyvinyl styrene, styrene-divinylbenzenze copolymers, polypropylene, polyethylene, polyethylene terephthalate, polytetrafluoroethylene (PTFE) and/or other polymers, or consist of these. The support material that can be used is, depending on the enzyme to be immobilized, also acidic or basic ion exchanger resins, for example Duolite A568, Duolite XAD 761, Duolite XAD 1180, Duolite XAD 7HP, Amberlite IR 120, Amberlite IR 400, Amberlite CG 50, Amberlyst 15 (all products from Rohm and Haas), Lewatit CNP 105 and (products from Lanxess, Leverkusen, Germany). Preferred supports are polypropylene supports or acrylate supports. Of particular preference are the polypropylene support Accurel MP1000 or the acrylate support Lewatit VP OC 1600. A preferred commercially available enzyme is a preparation of lipase B from Candida antarctica (CAL-B) (Novozyme® 435) immobilized on a polymethacrylate support.

Inorganic supports that can be used are oxidic and/or ceramic supports known from the prior art. In particular, inorganic supports that can be used are Celite, zeolite, silica, controlled-pore glass (CPG) or other supports.

Preferably, the support used has a particle size distribution in which at least 90% of the particles have a particle size of from 0.5 to 5000 μm, preferably from 1 to 2000 μm, particularly preferably from 10 to 2000 μm, in particular 25 to 2000 μm.

The process described here for the enzymatic esterification preferably takes place in a reactor which comprises at least one heterogeneous catalyst. The heterogeneous catalyst is preferably in the form of a fixed bed or is located suspended in the reaction mixture.

It is an advantage of the fixed bed that the supported enzyme does not have to be separated off separately from the product and the support particles are not able to come into contact with a stirrer. The particles thus remain better intact. However, it is disadvantageous that the reactants in most cases have to be passed several times over such a fixed bed in order to achieve an acceptable reaction conversion. Moreover, the removal of water of reaction can be more difficult, a pressure loss over the fixed bed can result or separation phenomena can arise.

In the suspension reactor, the reaction mixture is usually mixed with the help of a stirring device. Should this be the case, then the support particles are subjected to a certain mechanical stress. Further, there are options to dispense with a stirring device via the choice of reactor geometry and to ensure the required mixing in another way (e.g. by using a bubble column or an airlift reactor). An advantage of the suspension reactor is a reaction conversion in one step. Here, when using a heterogeneous catalyst, the separation of the reaction product by size exclusion methods is advantageously possible, e.g. using a sieve, with the help of which solids can be separated off according to the criterion of particle size. Specifically, the sieve method can also be a filtration. The size and/or geometry of the openings of the separation medium (for example filter pores) is oriented here to the smallest particle size to be separated off. Moreover, such a separation can also take place sequentially by means of serial connection of two or more different sieves and/or filters (different e.g. in the size and/or geometry of the openings of the separation medium). The selection often orients itself to the fact that a separation takes place as far as possible without pressure loss.

An advantage of keeping the supported enzyme separate from the reaction mixture (e.g. by means of fixed bed) or the ability of the supported enzyme to be separated off from suspensions is that the enzymes can be used several times in this way. The recovery of the enzyme in the form of the supported variant (as heterogeneous catalyst) is also preferred for this reason.

Preferably, the enzymatic esterification is carried out without the addition of an external solvent (termed “solvent-free” here).

During the ester synthesis, water of reaction is formed which leads to an undesired shift in the equilibrium of the reaction. Preferably, the water formed during the reaction is removed by customary processes known to the person skilled in the art. The amount of discharged water of reaction is chosen such that the reaction equilibrium is adequately shifted with regard to the desired product (ester). In other words, the conversion of the starting materials (alcohol and acid) should be as complete as possible in order to achieve the maximum possible ester yield. The desired conversion is generally greater than 80%, preferably greater than 85%, particularly preferably greater than 90%, in particular greater than 92%, in particular greater than 94%, in particular greater than 96%, in particular greater than 98%, in particular greater than 99%, in particular greater than 99.2%, in particular greater than 99.4%, in particular greater than 99.6%, in particular greater than 99.7%, in particular greater than 99.8%.

One option used for quantifying the reaction conversion is the measurement of the acid number, which is a measure of the acid present in the reaction mixture which has not reacted to give the ester. The person skilled in the art is familiar with this measurement.

The removal of the water of reaction can take place by means of the following measures:
distillative removal of the water,
use of a stripping or entrainer gas,
use of at least one drying agent,
   or a combination of at least two of these measures.

Suitable stripping or entrainer gases are e.g. air, nitrogen, carbon dioxide, argon or mixtures thereof. Suitable drying agents are e.g. molecular sieves, sodium sulfate, magnesium sulfate or silica gel.

Distillative removal of the water can take place at atmospheric pressure (about 1 atm=1013.25 mbar). It can also be carried out at pressures above or below atmospheric pressure. Reduced pressure (based on atmospheric pressure) is understood as meaning for example pressure in the range from 1 to 1000 mbar. Preference is given to using pressure ranges from 5 to 500 mbar, in particular 5 to 200 mbar, in particular 5 to 100 mbar, in particular 10 to 100 mbar, in particular 10 to 50 mbar. It is essential that the operating pressures at the respective reaction temperature are lower than the vapor pressure of the water of reaction.

Since the stability of enzymes is only ensured in certain temperature ranges, the reaction temperature must be chosen carefully. It is typically 0 to 100° C., preferably 20 to 100° C., particularly preferably 20 to 90° C. As mentioned above, the chosen reaction temperature brings about the vacuum that is to be applied optionally for the discharge of the water of reaction.

With the use of stripping or entrainer gases, discharge of water of reaction can likewise take place. Particularly in combination with the distillative removal of water of reaction, it is possible to achieve results which in particular have a positive effect on the yield in the esterification process. The mass transfer area and the gas volume are critical here. Preference is given to those gases which react neither with the reactants nor the catalyst, the reactor materials, let alone the end product, i.e. are inert. The person skilled in the art is familiar with this processing measure.

A specific suitable process for the enzymatic esterification is described in WO 2014/056756, to which reference is hereby made in its entirety.

The crude reaction mixtures obtained by the process according to the invention can be subjected to at least one work-up step. These include e.g. neutralization, purification and drying. Purification can take place by customary methods known to the person skilled in the art, e.g. by extraction and/or distillation.

As a rule, the process described above produces reaction products which, based on the compounds of the general formula (I) present, comprise pure diesters or mixtures of mono- and diesters.

A typical reaction product of the preparation of diesters comprises preferably 80 to 100% by weight of diesters and 0 to 20% by weight of monoesters, based on the total weight of the compounds (I).

A typical reaction product of the preparation of monoesters comprises preferably 40 to 80% by weight of monoesters and 20 to 60% by weight of diesters, based on the total weight of the compounds (I).

A typical reaction product of the preparation of diesters comprises preferably

69 to 97% by weight of diesters,
0 to 20% by weight of monoesters,
3 to 10% by weight of acid halides and/or carboxylic acids,
0 to 1% by weight of 2,5-di(hydroxymethyl)tetrahydrofuran,
based on the total weight of the reaction product.

A typical reaction product of the preparation of monoesters comprises preferably

29 to 77% by weight of monoesters,
20 to 60% by weight of diesters,
3 to 10% by weight of acid halides and/or carboxylic acids,
0 to 1% by weight of 2,5-di(hydroxymethyl)tetrahydrofuran,
based on the total weight of the reaction product.

The compounds of the general formula (I) are advantageously suitable as interface-active compounds. In particular, the compounds of the general formula (I) are suitable as surfactants, emulsifiers, solubilizers and foam formers.

Suitable surfactants are preferably compounds of the general formula (I), in which R² is hydrogen.

Suitable surfactants are preferably compounds of the general formula (I), in which R² is hydrogen and R¹ is unbranched or branched C₈-C₃₅-alkyl or unbranched or branched C₈-C₃₅-alkenyl with 1, 2, 3, 4, 5 or 6 double bonds, where C₈-C₃₅-alkyl and C₈-C₃₅-alkenyl can be substituted by 1, 2 or 3 hydroxyl groups and/or can have 1, 2, 3 or 4 epoxy groups.

Suitable surfactants are particularly preferably compounds of the general formula (I), in which R² is hydrogen and R¹ is unbranched or branched C₁₁-C₁₇-alkyl or unbranched or branched C₁₁-C₁₇-alkenyl with 1, 2, 3, 4, 5 or 6 double bonds, where C₁₁-C₁₇-alkyl radical and C₁₁-C₁₇-alkenyl can be substituted by 1, 2 or 3 hydroxyl groups.

Suitable surfactants are in particular compounds of the general formula (I), in which R² is hydrogen and R¹ is unsubstituted unbranched C₁₁-C₁₃-alkyl.

The aforementioned compounds (I) suitable as surfactants are also preferably suitable as foam formers. They are characterized by good foaming ability, i.e. with them it is firstly possible to achieve a good level of base foam, and on the other hand they also provide good foam stability, especially in hard water. As regards suitable and preferred foam formers, reference is made to the suitable and preferred surfactants in their entirety.

Suitable solubilizers are preferably compounds of the general formula (I), in which
R² is C(═O)R^{1′} and where at least one of the radicals R¹ and R^{1′} is unbranched or branched C₁₁-C₃₅-alkyl or unbranched or branched C₁₁-C₃₅-alkenyl with 1, 2, 3 or more than 3 double bonds, where C₁₁-C₃₅-alkyl and C₁₁-C₃₅-alkenyl can in each case be substituted by at least one hydroxyl group,
or
R² is hydrogen and R¹ is unbranched or branched C₁₁-C₃₅-alkyl or unbranched or branched C₁₁-C₃₅-alkenyl with 1, 2, 3 or more than 3 double bonds, where C₁₂-C₃₅-alkyl and C₁₁-C₃₅-alkenyl can be substituted by at least one hydroxyl group and/or can have at least one epoxy group.

In a first preferred embodiment, the solubilizers used are compounds of the general formula (I), in which
R² is C(═O)R^{1′} and where R¹ and R^{1′}, independently of one another, are unbranched or branched C₁₁-C₃₅-alkyl or unbranched or branched C₁₁-C₃₅-alkenyl with 1, 2, 3 or more than 3 double bonds, where C₁₁-C₃₅-alkyl and C₁₁-C₃₅-alkenyl can in each case be substituted by at least one hydroxyl group.

According to this first preferred embodiment, the solubilizers used are particularly preferably compounds of the general formula (I) in which
R² is C(═O)R^{1′} and where R¹ and R^{1′}, independently of one another, are unbranched or branched C₁₁-C₂₃-alkyl or unbranched or branched C₁₁-C₂₃-alkenyl with 1, 2, 3, 4, 5 or 6 double bonds, where C₁₁-C₂₃-alkyl and C₁₁-C₂₃-alkenyl can in each case be substituted by 1, 2 or 3 hydroxyl groups.

According to this first preferred embodiment, the solubilizers used are in particular compounds of the general formula (I) in which
R² is C(═O)R^{1′} and where R¹ and R^{1′}, independently of one another, are unbranched or branched C₁₅-C₂₁-alkyl or unbranched or branched C₁₅-C₂₁-alkenyl with 1, 2, 3 or 4 double bonds, where C₁₅-C₂₁-alkyl and C₁₅-C₂₁-alkenyl can in each case be substituted by 1, 2 or 3 hydroxyl groups.

In a second preferred embodiment, the solubilizers used are compounds of the general formula (I) in which
R² is C(═O)R^{1′} and where
R¹ is unbranched or branched C₁₁-C₃₅-alkyl or unbranched or branched C₁₁-C₃₅-alkenyl with 1, 2, 3 or more than 3 double bonds, where C₁₁-C₃₅-alkyl and C₁₁-C₃₅-alkenyl can in each case be substituted by at least one hydroxyl group, and
R^{1′} is unbranched or branched C₅-C₁₁-alkyl or unbranched or branched C₅-C₁₁-alkenyl with 1, 2 or 3 double bonds, where C₅-C₁₁-alkyl and C₅-C₁₁-alkenyl can in each case be substituted by at least one hydroxyl group.

According to this second preferred embodiment, the solubilizers used are particularly preferably compounds of the general formula (I) in which
R² is C(═O)R^{1′} and where
R¹ is unbranched or branched C₁₁-C₂₃-alkyl or unbranched or branched C₁₁-C₂₃-alkenyl with 1, 2, 3, 4, 5 or 6 double bonds, where C₁₁-C₂₃-alkyl and C₁₁-C₂₃-alkenyl can in each case be substituted by 1, 2 or 3 hydroxyl groups and/or can have 1, 2, 3 or 4 epoxy groups, and
R^{1′} is unbranched or branched C₅-C₁₁-alkyl or unbranched or branched C₅-C₁₁-alkenyl with 1 or 2 double bonds, where C₅-C₁₁-alkyl and C₅-C₁₁-alkenyl can in each case be substituted by 1 or 2 hydroxyl group.

According to this second preferred embodiment, the solubilizers used are in particular compounds of the general formula (I) in which
R² is C(═O)R^{1′} and where
R¹ is unbranched or branched C₁₅-C₂₁-alkyl or unbranched or branched C₁₅-C₂₁-alkenyl with 1, 2, 3 or 4 double bonds, where C₁₅-C₂₁-alkyl and C₁₅-C₂₁-alkenyl can in each case be substituted by 1, 2 or 3 hydroxyl groups, and
R^{1′} is unbranched or branched C₅-C₁₁-alkyl or unbranched or branched C₅-C₁₁-alkenyl with 1 or 2 double bonds, where C₅-C₁₁-alkyl and C₅-C₁₁-alkenyl can in each case be substituted by 1 or 2 hydroxyl group.

In a third preferred embodiment, the solubilizers used are compounds of the general formula (I) in which

R² is hydrogen and R¹ is unbranched or branched C₁₁-C₃₅-alkyl or unbranched or branched C₁₁-C₃₅-alkenyl with 1, 2, 3 or more than 3 double bonds, where C₁₁-C₃₅-alkyl and C₁₁-C₃₅-alkenyl can be substituted by at least one hydroxyl group and/or can have at least one epoxy group.

According to this third preferred embodiment, the solubilizers used are particularly preferably compounds of the general formula (I) in which
R² is hydrogen and R¹ is unbranched or branched C₁₁-C₂₃-alkyl or unbranched or branched C₁₁-C₂₃-alkenyl with 1, 2, 3, 4, 5 or 6 double bonds, where C₁₁-C₂₃-alkyl and C₁₁-C₂₃-alkenyl can be substituted by 1, 2 or 3 hydroxyl groups and/or can have 1, 2, 3 or 4 epoxy groups.

According to this third preferred embodiment, the solubilizers used are in particular compounds of the general formula (I) in which
R² is hydrogen and R¹ is unbranched or branched C₁₅-C₂₁-alkyl or unbranched or branched C₁₅-C₂₁-alkenyl with 1, 2, 3 or 4 double bonds, where C₁₅-C₂₁-alkyl and C₁₅-C₂₁-alkenyl can be substituted by 1, 2 or 3 hydroxyl groups.

The compounds of the general formula (I) are suitable in an advantageous manner for modifying the rheological properties of aqueous compositions. They may quite generally be for example cosmetic compositions, pharmaceutical compositions, hygiene products, coating compositions, compositions for the paper industry, and the textile industry.

The compounds of the general formula (I) are preferably suitable for thickening the consistency of surfactant-containing aqueous compositions within a wide range. They function here specifically as so-called “micellar thickeners”, i.e. interface-active compounds which are used for increasing the viscosity of surfactant-containing formulations. Depending on the basic structure of the liquid compositions, flow properties from thin-liquid ranging to solid (in the sense of “no longer flowable”) can generally be achieved depending on the use amount of the copolymer.

Suitable rheology modifiers are preferably compounds of the general formula (I) in which
R² is hydrogen and R¹ is unbranched or branched C₈-C₃₅-alkenyl with 1, 2, 3 or more than 3 double bonds, where C₈-C₃₅-alkenyl can be substituted by at least one hydroxyl group and/or can have at least one epoxy group.

Suitable rheology modifiers are particularly preferably compounds of the general formula (I) in which
R² is hydrogen and R¹ is unbranched or branched C₈-C₃₅-alkenyl with 1, 2, 3, 4, 5 or 6 double bonds, where C₈-C₃₅-alkenyl can be substituted by 1, 2 or 3 hydroxyl groups and/or can have 1, 2, 3 or 4 epoxy groups.

Suitable rheology modifiers are in particular compounds of the general formula (I) in which
R² is hydrogen and R¹ is unbranched or branched C₈-C₂₃-alkenyl with 1, 2, 3 or 4 double bonds, where C₈-C₂₃-alkenyl can be substituted by 1, 2 or 3 hydroxyl groups.

Suitable emollients are preferably compounds of the general formula (I) in which
R² is C(═O)R^{1′} and where R¹ and R^{1′}, independently of one another, are unbranched or branched C₈-C₂₃-alkyl or unbranched or branched C₈-C₂₃-alkenyl with 1, 2, 3 or more than 3 double bonds.

Particularly preferably, R² is C(═O)R^{1′}, and R¹ and R^{1′}, independently of one another, are branched C₈-C₁₄-alkyl or branched C₈-C₂₃-alkenyl with 1, 2 or 3 double bonds.

For the use as emollients, the radicals R¹ and R^{1′} are preferably unsubstituted.

Suitable emollients are particularly preferably compounds of the general formula (I) which have a melting point of at most 40° C., particularly preferably of at most 30° C.

Compositions According to the Invention which Comprise at Least One Additional Surfactant

The compounds of the general formula (I) according to the invention are particularly advantageously suitable for providing formulations, specifically cosmetic or pharmaceutical compositions, and also detergents, cleaners or dishwashing compositions which comprise at least one surfactant different from the compounds of the general formula (I). In particular, these are aqueous formulations. The compounds (I) are characterized in such formulations by at least one of the following properties: a good surface-active effect, a good thickening effect even for low use amounts, and a good compatibility with further surfactants.

Suitable additional surfactants are anionic surfactants, nonionic surfactants, cationic surfactants, amphoteric surfactants and mixtures thereof.

Typical examples of anionic surfactants are soaps, alkylsulfonates, alkylbenzenesulfonates, olefinsulfonates, alkyl ether sulfonates, glycerol ether sulfonates, methyl ester sulfonates, sulfo fatty acids, alkyl sulfates, ether sulfates, glycerol ether sulfates, fatty acid ether sulfates, hydroxyl mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as for example acyl glutamates and acyl aspartates, and also acyl lactylates, acyl tartrates, alkyl oligoglucoside sulfates, alkylglucose carboxylates, protein fatty acid condensates and alkyl (ether)phosphates.

Suitable soaps are e.g. alkali metal, alkaline earth metal and ammonium salts of fatty acids, such as potassium stearate.

Preferred alkyl sulfates are sulfates of fatty alcohols of the general formula R³—O—SO₃Y¹, in which R³ is a linear or branched, saturated or unsaturated hydrocarbon radical having 6 to 22 carbon atoms and Y¹ is an alkali metal, the monovalent charge equivalent of an alkaline earth metal, ammonium, mono-, di-, tri- or tetraalkylammonium, alkanolammonium or glucammonium. Suitable fatty alcohol sulfates are preferably obtained by sulfation of native fatty alcohols or synthetic oxo alcohols and subsequent neutralization. Typical examples of fatty alcohol sulfates are the sulfation products of caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, behenyl alcohol and elaeostearyl alcohol, and also the salts and mixtures thereof. Preferred salts of the fatty alcohol sulfates are the sodium and potassium salts, in particular the sodium salts. Preferred mixtures of the fatty alcohol sulfates are based on technical-grade alcohol mixtures which are produced e.g. during the high-pressure hydrogenation of technical-grade methyl esters based on fats and oils or during the hydrogenation of aldehydes from the oxo synthesis or during the dimerization of unsaturated fatty alcohols. For the preparation of alkyl sulfates, preference is given to using fatty alcohols and fatty alcohol mixtures having 12 to 18 carbon atoms and in particular 16 to 18 carbon atoms. Typical examples thereof are technical-grade alcohol sulfates based on plant raw materials.

Suitable olefin sulfonates are obtained e.g. by the addition reaction of SO₃ onto olefins of the formula R⁴—CH═CH—R⁵ and subsequent hydrolysis and neutralization, where R⁴ and R⁵, independently of one another, are H or alkyl radicals having 1 to 20 carbon atoms, with the proviso that R⁴ and R⁵ together have at least 6 and preferably 8 to 20, specifically 10 to 16, carbon atoms. The olefin sulfonates can be present as alkali metal, alkaline earth metal, ammonium, alkylammonium, alkanolammonium or glucammonium salts. The olefin sulfonates are preferably present as sodium salts. The hydrolyzed alpha-olefin sulfonation product, i.e. the alpha-olefin sulfonates, are composed of about 60% by weight of alkane sulfonates and about 40% by weight of hydroxyalkane sulfonates; of this, about 80 to 85% by weight are monosulfonates and 15 to 20% by weight are disulfonates.

Preferred methyl ester sulfonates (MES) are obtained by sulfonation of the fatty acid methyl esters of plant or animal fats or oils. Preference is given to methyl ester sulfonates from plant fats and oils, e.g. from rapeseed oil, sunflower oil, soyabean oil, palm oil, coconut fat, etc.

A preferred class of anionic surfactants is the ether sulfates. Ether sulfates (alkyl ether sulfates) are known anionic surfactants which can be prepared industrially by SO₃— or chlorosulfonic acid (CSA)-sulfation of fatty alcohol or oxo alcohol polyglycol ethers and subsequent neutralization.

Preference is given in particular to fatty alcohol ether sulfates of the general formula R⁶O—(CH₂CH₂O)ₘSO₃Y², in which R⁶ is a linear or branched alkyl and/or alkenyl radical having 6 to 22 carbon atoms, m is numbers from 1 to 10 and r is an alkali metal and/or alkaline earth metal, ammonium, alkylammonium, alkanolammonium or glucammonium. Typical examples are the sulfates of addition products of on average 1 to 10 and in particular 2 to 5 mol of ethylene oxide onto caprolic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and technical-grade mixtures thereof in the form of their sodium and/or magnesium salts. The ether sulfates here can have either a conventional or a narrowed homolog distribution. Particular preference is given to the use of ether sulfates based on adducts of on average 2 to 3 mol of ethylene oxide onto technical-grade C_{12/14}- or C_{12/18}-coconut fatty alcohol fractions in the form of their sodium and/or magnesium salts.

Preferred sarcosinates are sodium lauroyl sarcosinate or sodium stearoyl sarcosinate.

Preferred protein fatty acid condensates are plant products based on wheat.

Preferred alkyl phosphates are mono- and diphosphoric acid alkyl esters.

Suitable acyl glutamates are compounds of the formula (II)

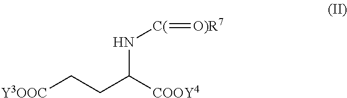

in which COR⁷ is a linear or branched acyl radical having 6 to 22 carbon atoms and 0, 1, 2 or 3 double bonds and Y³ and Y⁴, independently of one another, are hydrogen, an alkali metal, the monovalent charge equivalent of an alkaline earth metal, ammonium, alkylammonium, alkanolammonium or glucammonium. The preparation of acyl glutamates takes place for example by Schotten-Baumann acylation of glutamic acid with fatty acids, fatty acid esters or fatty acid halides. Acyl glutamates are commercially available for example from BASF SE, Clariant AG, Frankfurt/DE, or Ajinomoto Co. Inc., Tokyo/JP. An overview of the preparation and properties of acyl glutamates can be found by M. Takehara et al. in J. Am. Oil Chem. Soc. 49 (1972) 143. Typical acyl glutamates suitable as component b) are preferably derived from fatty acids having 6 to 22 and particularly preferably 12 to 18 carbon atoms. In particular, the mono- or dialkali metal salts of the acyl glutamate are used. These include e.g. (trade names of Ajinomoto, USA in brackets): sodium cocoyl glutamate (Amisoft CS-11), disodium cocoyl glutamate (Amisoft ECS-22SB), triethanolammonium cocoyl glutamate (Amisoft CT-12), triethanolammonium lauroyl glutamate (Amisoft LT-12), sodium myristoyl glutamate (Amisoft MS-11), sodium stearoyl glutamate (Amisoft HS-11 P) and mixtures thereof.

The nonionic surfactants include for example:
fatty alcohol polyoxyalkylene esters, for example lauryl alcohol polyoxyethylene acetate,
alkyl polyoxyalkylene ethers which are derived from low molecular weight C₁-C₆-alcohols or from C₇-C₃₀-fatty alcohols. Here, the ether component can be derived from ethylene oxide units, propylene oxide units, 1,2-butylene oxide units, 1,4-butylene oxide units and random copolymers and block copolymers thereof. These include specifically fatty alcohol alkoxylates and oxo alcohol alkoxylates, in particular of the type RO—(R⁸O)ᵣ(R⁹O)ₛR¹⁰ where R⁸ and R⁹, independently of one another, ═C₂H₄, C₃H₆, C₄H₈ and R¹⁰=H, or C₁-C₁₂-alkyl, R=C₃-C₃₀-alkyl or C₆-C₃₀-alkenyl, r and s, independently of one another, are 0 to 50, where both cannot be 0, such as isotridecyl alcohol and oleyl alcohol polyoxyethylene ether,
alkylaryl alcohol-polyoxyethylene ether, e.g. octylphenol-polyoxyethylene ether,
sugar surfactants, preferably alkyl polyglycosides, sorbitol esters, such as for example sorbitan fatty acid esters (sorbitan monooleate, sorbitan tristearate), polyoxyethylene sorbitan fatty acid esters, N-alkylgluconamides,
alkoxylated animal and/or vegetable fats and/or oils, for example corn oil ethoxylates, castor oil ethoxylates, tallow fatty ethoxylates,
glycerol esters, such as, for example, glycerol monostearate,
alkylphenol alkoxylates, such as for example ethoxylated isooctyl-, octyl- or nonylphenol, tributylphenol polyoxyethylene ether,
fatty amine alkoxylates, fatty acid amide and fatty acid diethanolamide alkoxylates, in particular ethoxylates thereof,
alkylmethyl sulfoxides,
alkyldimethylphosphine oxides, such as for example tetradecyldimethylphosphine oxide.

A preferred class of nonionic surfactants is the sugar surfactants, specifically alkyl (poly)glycosides. In the context of the invention, the term alkyl (poly)glycosides is used synonymously to alkyl (oligo)glycosides and is also referred to by the abbreviation “APG”. Alkyl glycosides and/or alkyl polyglycosides comprise both alkyl and also alkenyl (poly)glycosides and preferably have the formula R¹¹O-[G]ₚ, in which R¹¹ is an alkyl and/or alkenyl radical having 4 to 22 carbon atoms, G is a sugar radical having 5 or 6 carbon atoms and p is numbers from 1 to 10. They can be obtained by the relevant methods of preparative organic chemistry. The alkyl and/or alkenyl oligoglycosides can be derived from aldoses or ketoses having 5 or 6 carbon atoms, preferably glucose. The preferred alkyl and/or alkenyl oligoglycosides are therefore alkyl and/or alkenyl oligoglucosides. The index number p gives the degree of polymerization (DP), i.e. the distribution of mono- and oligoglycosides, and stands for a number between 1 and 10. Whereas p in a given compound must always be an integer and here in particular can assume the values p=1 to 6, the value p for a specific alkyl polyglycoside is an analytically determined calculated parameter which in most cases is a fraction. Preference is given to using alkyl and/or alkenyl polyglycosides with an average degree of polymerization p of 1.1 to 3.0. From an applications point of view, preference is given to those alkyl and/or alkenyl polyglycosides whose degree of polymerization is less than 1.7 and is in particular between 1.2 and 1.4.

Suitable amphoteric surfactants are e.g. alkylbetaines, alkylamidopropylbetaines, alkylsulfobetaines, alkylglycinates, alkyl carboxyglycinates, alkyl amphoacetates or -propionates, alkyl amphodiacetates or -dipropionates. For example, cocodimethylsulfopropylbetaine, laurylbetaine, cocamidopropylbetaine, sodium cocamphopropionate or tetradecyldimethylamine oxide can be used.

The cationic surfactants include, for example, quaternized ammonium compounds, in particular alkyltrimethylammonium and dialkyldimethylammonium halides and alkyl sulfates, and also pyridine and imidazoline derivatives, in particular alkylpyridinium halides. For example, behenyl or cetyltrimethylammonium chloride can be used. Also of suitability are so-called ester quats, which are based on quaternary triethanol-methyl-ammonium or quaternary diethanol-dimethyl-ammonium compounds with long hydrocarbon chains in the form of fatty acid esters. These include, for example, bis(acyloxyethyl)hydroxyethylammonium methosulfate. Also of suitability is dehyquart L 80 (INCI: Dicocoylethyl Hydroxyethylmonium Methosulfate (and) Propylene Glycol).

### Cosmetic and Pharmaceutical Compositions

The compounds of the general formula (I) are preferably suitable for formulating cosmetic and pharmaceutical compositions, specifically aqueous cosmetic and pharmaceutical compositions.

A further subject of the invention is accordingly a cosmetic or pharmaceutical composition which comprises at least one compound of the general formula (I), as defined above, and at least one cosmetic or pharmaceutical active ingredient and/or auxiliary different therefrom.

Preferably, the cosmetic and pharmaceutical compositions according to the invention comprise the compounds of the general formula (I) in an amount from 0.1 to 50% by weight, particularly preferably from 0.5 to 30% by weight, based on the total weight of the composition.

Preferably, the cosmetic and pharmaceutical compositions according to the invention comprise at least one cosmetically or pharmaceutically acceptable carrier as auxiliary.

Preferably, the cosmetically or pharmaceutically acceptable carrier is selected from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di-, or trihydric alcohols different from iii),
v) saturated acyclic and cyclic hydrocarbons,
vi) fatty acids,
vii) fatty alcohols,
viii) propellant gases,
and mixtures thereof.

Specifically suitable cosmetically compatible oils, fats and waxes are described in Karl-Heinz Schrader, Grundlagen and Rezepturen der Kosmetika [Fundamentals and Formulations of Cosmetics], 2nd edition, Verlag Hüthig, Heidelberg, pp. 319-355 (1989), to which reference is made here.

Preferred oils, fats and waxes are mineral and synthetic oils, such as e.g. paraffins and aliphatic hydrocarbons with more than 8 carbon atoms, purcellin oil, perhydrosqualene, silicone oils, natural (animal or plant) oils and fats, such as e.g. sunflower oil, coconut oil, palm kernel oil, palm oil, soyabean oil, avocado oil, olive oil, sweet almond oil, calophylum oil, castor oil, sesame oil, jojoba oil, carite oil, hoplostethus oil, lanolin and derivatives thereof (e.g. hydrogenated lanolin and acetylated lanolin), or waxes, fatty acids, fatty acid esters such as e.g. triglycerides of C₆-C₃₀-fatty acids, wax esters, fatty alcohols, Vaseline, and mixtures thereof. Suitable waxes are e.g. carnauba wax, candililla wax, beeswax, microcrystalline wax, ozocerite wax and Ca, Mg and Al oleates, myristates, linoleates and stearates. Such cosmetically compatible oils, fats and waxes are used especially in skin cosmetic and dermatological compositions.

The compositions according to the invention can be skin cosmetic, hair cosmetic, dermatological, hygiene or pharmaceutical compositions. On account of the properties described above, the compounds of the general formula (I) are advantageously suitable for use in a large number of different cosmetic or pharmaceutical compositions. They can be used here as active ingredient, as auxiliary or as a component with a multiple action. Thus, the compounds (I) are suitable e.g. in the area of skin and hair cleansing for compositions which have at least one of the following properties: a good cleaning performance; good rheological properties, i.e. although the compositions are flowable, they are not too liquid or viscous in order to permit an optimum application; the ability to supply the skin with moisture or to have a refatting effect.

The compounds (I) are also preferably suitable as foam formers in cosmetic compositions, in particular compositions for the care and/or cleansing of the hair.

In a specific embodiment, the compositions according to the invention are a cleansing composition for the skin and/or the hair. These comprise preferably at least one of the compounds of the formula (I) described above with the suitability as surfactants.

In a further specific embodiment, the compositions according to the invention are compositions for the care and/or protection of the skin. These can then comprise at least one of the above-described compounds of the formula (I) with the suitability as emollients. These can furthermore comprise at least one sparingly soluble active ingredient, e.g. a UV filter, and at least one of the above-described compounds of the formula (I) with the suitability as solubilizer.

In a further specific embodiment, the compositions according to the invention are a composition for decorative cosmetics. A further specific embodiment is skin and hair conditioners which comprise at least one compound of the formula (I).

On account of their thickening properties, the above-described compounds of the formula (I) are in particular suitable also as additives for hair and skin cosmetics.

Preferably, the compositions according to the invention are present in the form of a cream, mousse, milk, lotion, mascara, stage makeup, soap of liquid to solid consistency, foam, gel, spray, stick, washing, showering or bathing preparation of liquid to gel-like consistency, eyeshadow, eyeliner, blusher, powder or strip. If desired, liposomes or microspheres can also be used.

The cosmetic compositions according to the invention can additionally comprise cosmetically and/or dermatologically active ingredients and effect substances and also auxiliaries. Preferably, the cosmetic compositions according to the invention comprise at least one compound of the formula (I), as defined above, at least one carrier c) as defined above and at least one constituent different therefrom which is preferably selected from cosmetically active ingredients, emulsifiers, surfactants, preservatives, perfume oils, additional thickeners, hair polymers, hair and skin conditioners, graft polymers, water-soluble or dispersible silicone-containing polymers, light protection agents, bleaches, gel formers, care agents, tinting agents, tanning agents, dyes, pigments, consistency regulators, humectants, refatting agents, collagen, protein hydrolysates, lipids, antioxidants, antifoams, antistats, emollients and softeners.

In addition to the compounds of the formula (I), the cosmetic compositions can comprise at least one conventional thickener. These include e.g. polysaccharides and organic sheet minerals such as Xanthan Gum® (Kelzan® from Kelco), Rhodopol® 23 (Rhone Poulenc) or Veegum® (R. T. Vanderbilt) or Attaclay® (Engelhardt). Suitable thickeners are also organic natural thickeners (agar agar, carrageenan, tragacanth, gum Arabic, alginates, pectins, polyoses, guar flour, carob seed flour, starch, dextrins, gelatins, casein) and inorganic thickeners (polysilicic acids, clay minerals such as montmorillonites, zeolites, silicas).

Suitable cosmetically and/or dermatologically active ingredients are e.g. skin and hair pigmentation agents, tanning agents, bleaches, keratin-hardening substances, antimicrobial active ingredients, light filter active ingredients, repellant active ingredients, hyperemic substances, keratolytically and keratoplastically acting substances, antidandruff active ingredients, antiphlogistics, keratinizing substances, active ingredients with an antioxidative or free-radical scavenging effect, skin-moisturizing or -humectant substances, refatting active ingredients, deodorizing active ingredients, sebostatic active ingredients, plant extracts, antierythematous or antiallergic active ingredients and mixtures thereof.

Artificially skin-tanning active ingredients which are suitable for tanning the skin without natural or artificial irradiation with UV rays are e.g. dihydroxyacetone, alloxan and walnut shell extract. Suitable keratin-hardening substances are generally active ingredients which are also used in antiperspirants, such as e.g. potassium aluminum sulfate, aluminum hydroxychloride, aluminum lactate, etc. Antimicrobial active ingredients are used in order to destroy microorganisms and/or to inhibit their growth and thus serve both as preservatives and also as deodorizing substance which reduces the formation or the intensity of body odor. These include e.g. customary preservatives known to the person skilled in the art, such as p-hydroxybenzoic acid esters, imidazolidinylurea, formaldehyde, sorbic acid, benzoic acid, salicylic acid, etc. Such deodorizing substances are e.g. zinc ricinoleate, triclosan, undecylenic acid alkylolamides, citric acid triethyl esters, chlorhexidine, etc. Suitable light filter active ingredients are substances which absorb UV rays in the UV-B and/or UV-A region. Suitable UV filters are those mentioned above. Also of suitability are p-aminobenzoic acid esters, cinnamic acid esters, benzophenones, camphor derivatives, and pigments that deflect UV rays, such as titanium dioxide, talc and zinc oxide. Suitable repellant active ingredients are compounds which are able to deter or to repel certain animals, in particular insects, from people. These include e.g. 2-ethyl-1,3-hexanediol, N,N-diethyl-m-toluamide, etc. Suitable hyperemic substances which stimulate circulation in the skin are e.g. essential oils, such as dwarf pine, lavender, rosemary, juniper berry, horsechestnut extract, birch leaf extract, hayflower extract, ethyl acetate, camphor, menthol, peppermint oil, rosemary extract, eucalyptus oil, etc. Suitable keratolytically and keratoplastically acting substances are e.g. salicylic acid, calcium thioglycolate, thioglycolic acid and its salts, sulfur, etc. Suitable antidandruff active ingredients are e.g. sulfur, sulfur polyethylene glycol sorbitan monooleate, sulfur ricinol polyethoxylate, zinc pyrithione, aluminum pyrithione, etc. Suitable antiphlogistics, which counteract skin irritations, are e.g. allantoin, bisabolol, dragosantol, chamomile extract, panthenol, etc.

The cosmetic compositions according to the invention can comprise, as cosmetic active ingredient (and also optionally as auxiliary), at least one cosmetically or pharmaceutically acceptable polymer. These include quite generally anionic, cationic, amphoteric and neutral polymers.

According to a preferred embodiment, the compositions according to the invention are a skin cleansing composition.

Preferred skin cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, showering and bathing preparations, such as washing lotions, shower baths and gels, foam baths, oil baths and scrub preparations, shaving foams, lotions and creams.

According to a further preferred embodiment, the compositions according to the invention are cosmetic compositions for the care and protection of the skin, nail care compositions or preparations for decorative cosmetics.

Suitable skin cosmetic compositions are e.g. face tonics, face masks, deodorants and other cosmetic lotions. Compositions for use in decorative cosmetics comprise for example concealing sticks, stage makeup, mascara and eyeshadows, lipsticks, kohl pencils, eyeliners, blushers, powders and eyebrow pencils.

Furthermore, the compounds of the formula (I) can be used in nose strips for pore cleansing, in antiacne compositions, repellants, shaving compositions, hair removal compositions, intimate care compositions, foot care compositions, and also in baby care.

The skincare compositions according to the invention are in particular W/O or O/W skin creams, day and night creams, eye creams, face creams, antiwrinkle creams, moisturizing creams, bleaching creams, vitamin creams, skin lotions, care lotions and moisturizing lotions.

Skin cosmetic and dermatological compositions based on the above-described compounds of the formula (I) exhibit advantageous effects. The compounds of the formula (I) can contribute inter alia to the moisturization and conditioning of the skin and to the improvement in skin feel. By adding the polymers according to the invention, in certain formulations it is possible to achieve a considerable improvement in skin compatibility.

Skin cosmetic and dermatological compositions comprise preferably at least one compounds of the formula (I) in a fraction of about 0.001 to 30% by weight, preferably 0.01 to 20% by weight, very particularly preferably 0.1 to 12% by weight, based on the total weight of the composition.

Depending on the field of use, the compositions according to the invention can be applied in a form suitable for skincare, such as e.g. as cream, foam, gel, stick, mousse, milk, spray (pump spray or propellant-containing spray) or lotion.

Besides the compounds of the formula (I) and suitable carriers, the skin cosmetic compositions can also comprise further active ingredients and auxiliaries customary in skin cosmetics, as described above. These include preferably emulsifiers, preservatives, perfume oils, cosmetic active ingredients such as phytantriol, vitamin A, E and C, retinol, bisabolol, panthenol, light protection agents, bleaching agents, tanning agents, collagen, protein hydrolysates, stabilizers, pH regulators, dyes, salts, thickeners, gel formers, consistency regulators, silicones, humectants, refatting agents and further customary additives.

In order to establish certain properties, such as e.g. improving the feel to touch, the spreading behavior, the water resistance and/or the binding of active ingredients and auxiliaries, such as pigments, the skin cosmetic and dermatological compositions can additionally also comprise conditioning substances based on silicone compounds. Suitable silicone compounds are, for example, polyalkylsiloxanes, polyarylsiloxanes, polyarylalkylsiloxanes, polyethersiloxanes or silicone resins.

The preparation of the cosmetic or dermatological compositions takes place by customary processes known to the person skilled in the art.

According to a further preferred embodiment, the compositions according to the invention are a shower gel, a shampoo formulation or a bathing preparation. Such formulations comprise at least one compound of the general formula (I), and usually anionic surfactants as base surfactants and amphoteric and/or nonionic surfactants as cosurfactants. Further suitable active ingredients and/or auxiliaries are generally selected from lipids, perfume oils, dyes, organic acids, preservatives and antioxidants, and also thickeners/gel formers, skin conditioning agents and humectants. Suitable surfactants are those mentioned above.

According to a further preferred embodiment, the compositions according to the invention are a hair treatment composition. Preferably, the hair treatment compositions according to the invention are present in the form of a foam setting composition, hair mousse, hair gel, shampoo, hairspray, hair foam, end fluid, neutralizer for permanent waves or “hot-oil treatments”. Depending on the field of use, the hair cosmetic preparations can be applied as (aerosol) spray, (aerosol) foam, gel, gel spray, cream, lotion or wax.

The compounds of the formula (I) according to the invention and used according to the invention are likewise suitable for use for the modification of rheological properties in pharmaceutical compositions of every type. A further subject of the invention is the use of a compound of the formula (I), as defined above, as auxiliary in pharmacy.

Typical pharmaceutical compositions comprise
A) at least one compound of the formula (I), as defined above,
B) at least one pharmaceutically acceptable active ingredient and
C) optionally at least one further pharmaceutically acceptable auxiliary different from A) and B).

Pharmaceutically acceptable auxiliaries C) are the auxiliaries that are known for use in the field of pharmacy, food technology and related fields, in particular the auxiliaries listed in relevant Pharmacopeia (e.g. DAB, Ph. Eur., BP, NF), and also others whose properties do not preclude a physiological application.

Suitable auxiliaries C) can be: glidants, wetting agents, emulsifying and suspending agents, preserving agents, antioxidants, antiirritatives, chelating agents, emulsion stabilizers, film formers, gel formers, odor masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilizing agents, propellants, drying agents, opacifiers, additional thickeners, waxes, softeners, white oils. An embodiment with regard to this is based on expert knowledge.

To produce pharmaceutical compositions according to the invention, the active ingredients can be mixed or diluted with a suitable excipient. Excipients can be solid, semisolid or liquid materials which can serve as vehicles, carriers or medium for the active ingredient. The admixing of further auxiliaries takes place if desired in the manner known to the person skilled in the art. In particular, these are aqueous solutions or solubilizates for oral or for parenteral application. Furthermore, the copolymers to be used according to the invention are also suitable for use in oral administration forms such as tablets, capsules, powders, solutions. Here, they can provide the sparingly soluble medicament with an increased bioavailability. In the case of parenteral application, emulsions, for example fatty emulsions, can also be used besides solubilizates.

Pharmaceutical compositions of the type mentioned above can be obtained by processing the compounds of the formula (I) b be used according to the invention with pharmaceutical active ingredients by conventional methods and using known and new active ingredients.

The content of at least one compound of the general formula (I) is present in the pharmaceutical compositions, depending on the active ingredient, in the range of 0.01 to 50% by weight, preferably 0.1 to 40% by weight, particularly preferably 1 to 30% by weight, based on the total weight of the composition.

Of suitability for preparing the pharmaceutical compositions according to the invention are in principle all pharmaceutical active ingredients and prodrugs. These include benzodiazepines, antihypertensives, vitamins, cytostatics—in particular taxol, anesthetics, neuroleptics, antidepressants, antibiotics, antimycotics, fungicides, chemotherapeutics, urologics, platelet aggregation inhibitors, sulfonamides, spasmolytics, hormones, immunglobulins, sera, thyroid therapeutics, psychopharmaceuticals, anti Parkinson's agents and other antihyperkinetics, ophthalmics, neuropathy products, calcium metabolism regulators, muscle relaxants, narcotics, lipid-lowering agents, liver therapeutics, coronary agents, cardiac agents, immunotherapeutics, regulatory peptides and their inhibitors, hypnotics, sedatives, gynecologicals, gout remedies, fibrinolytics, enzyme preparations and transport proteins, enzyme inhibitors, emetics, perfusion promoters, diuretics, diagnostics, corticoids, cholinergics, biliary therapeutics, antiasthmatics, broncholytics, beta-receptor blockers, calcium antagonists, ACE inhibitors, arteriosclerosis agents, antiphlogistics, anticoagulants, antihypotensives, antihypoglycaemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, and weight-reduction agents. Examples of suitable pharmaceutical active ingredients are in particular the active ingredients specified in paragraphs 0105 to 0131 of US 2003/0157170.

### Detergents, Cleaners and Dishwashing Compositions

The compounds of the general formula (I) are advantageously suitable for use in detergents and cleaners, in dishwashing compositions and in rinse aids.

Examples of cleaners which comprise the compounds of the general formula (I) comprise detergents and cleaners, dishwashing compositions, such as hand dishwashing compositions or machine dishwashing compositions (=dishwashing compositions for dishwashers), metal degreasers, glass cleaners, floor cleaners, all-purpose cleaners, high-pressure cleaners, neutral cleaners, alkaline cleaners, acid cleaners, spray degreasers, dairy cleaners, commercial kitchen cleaners, apparatus cleaners in industry, in particular the chemical industry, cleaners for carwashes and also household all-purpose cleaners.

A further subject of the invention is detergents, cleaners and dishwashing compositions which comprise at least one compound of the general formula (I). Here, the compound of the general formula (I) can be used either as interface-active compound or else as rheology modifier. Reference is made here to the above-described suitable and preferred compounds (I) for the use as interface-active compound and as rheology modifier in their entirety.

The detergent, cleaner and dishwashing composition according to the invention preferably comprises the following constituents:
a) at least one compound of the general formula (I);
b) at least one builder (also referred to as sequestering agent, builder substance, complexing agent, chelator, chelating agent or softener);
c) optionally at least one enzyme;
d) optionally at least one bleach; and
e) optionally at least one further additive which is preferably selected from surfactants different from a), bases, corrosion inhibitors, antifoams, dyes, fragrances, fillers, tableting auxiliaries, disintegrants, thickeners, solubility promoters, organic solvents, electrolytes, pH extenders, perfume carriers, fluorescent agents, hydrotropes, antiredeposition agents, optical brighteners, graying inhibitors, shrink preventers, anticrease agents, color transfer inhibitors, antimicrobial active ingredients, antioxidants, corrosion inhibitors, antistats, ironing aids, phobicization and impregnation agents, swelling and slip-resistant agents, UV absorbers and water.

Preferably, the detergents and cleaners according to the invention comprise:
a) at least one compound of the general formula (I): from 0.1 to 20% by weight;
b) at least one builder: from 5 to 80% by weight;
c) at least one enzyme: from 0 to 8% by weight;
d) at least one bleach: from 0 to 30% by weight; and
e) at least one further additive: 0 to 50% by weight.

The % by weight data refer here to the total weight of the detergent and cleaner. The weight amounts from a) to e) add up to 100% by weight.

A dishwashing composition according to the invention preferably comprises the following constituents:
a) at least one compound of the general formula (I),
b) at least one builder (also referred to as sequestering agent, builder substance, complexing agent, chelator, chelating agent or softener),
c) optionally at least one enzyme,
d) optionally at least one bleach,
e1) water,
e2) optionally at least one thickener, and
e3) optionally at least one further additive which is preferably selected from surfactants different from a), bases, corrosion inhibitors, antifoams, dyes, fragrances, fillers, solubility promoters and organic solvents.

The composition according to the invention for dishwashing comprises, based on the total weight of the composition, preferably:
a) 0.1 to 50% by weight of at least one compound of the formula (I) according to the invention,
b) 5 to 90% by weight of at least one builder and/or cobuilder,
c) 0 to 8% by weight of at least one enzyme,
d) 0 to 30% by weight of at least one bleach,
e1) 0.1 to 90% by weight of water,
e2) 0 to 8% by weight of at least one thickener,
e3) 0 to 25% by weight of at least one further additive,
   with the proviso that the amounts by weight of components a) to e) add up to 100% by weight.

Moreover, the compounds of the formula (I) are suitable for producing aqueous preparations of food supplements such as water-insoluble vitamins and provitamins such as vitamin A, vitamin A acetate, vitamin D, vitamin E, tocopherol derivatives such as tocopherol acetate and vitamin K.

In general, the compounds of the general formula (I) according to the invention can be used in all areas where a thickening effect is necessary in combination with interface-active substances.

Furthermore, the compounds of the general formula (I) are suitable for improving the solubility of other components, e.g. of other surface-active components, such as of anionic surfactants. They therefore also make a positive contribution to the formation of clear surfactant-containing solutions.

The compounds of the general formula (I) are also particularly suitable as solubilizers for sparingly soluble substances. Specifically, these are sparingly soluble substances which have a solubility in water below 10 g/l at 25° C. and 1013 mbar.

Active ingredients for cosmetics, medicaments, crop protection and for material protection, i.e. substances which already develop an effect even at low concentration, e.g. a cosmetic effect, a pharmacological effect in an organism, a physiological effect in a plant or a harmful organism, etc., are often formulated and used in the form of liquid, specifically aqueous, compositions. Alternatively, a formulation and administration in solid form, e.g. as powder or compact (tablet, etc.), is also possible, in which case transportation to the actual site of action, however, comprises the conversion to a liquid, specifically aqueous, form.

Surprisingly, it has now been found that the compounds of the general formula (I) are suitable as solubilizers for a large number of sparingly soluble substances.

The compositions of water-insoluble substances comprise preferably an aqueous medium as continuous phase, at least one substance dispersed or solubilized in the continuous phase and having a solubility in water at 25° C./1013 mbar of less than 10 g/l, in particular less than 1 g/l and specifically less than 0.1 g/l, and also at least one compound of the general formula (I) according to the invention.

The term “aqueous medium” comprises in the context of the invention water and mixtures of water with organic solvents which are at least partially miscible with water. Examples of water-miscible solvents comprise C₃-C₄-ketones, such as acetone and methyl ethyl ketone, cyclic ethers, such as dioxane and tetrahydrofuran, C₁-C₄-alkanols, such as methanol, ethanol, n-propanol, isopropanol, n-butanol, tert-butanol, polyols and mono- and dimethyl ethers thereof, such as glycol, propanediol, ethylene glycol monomethyl ether, diethylene glycol, diethylene glycol monomethyl ether, diethylene glycol dimethyl ether, glycerol, also C₂-C₃-nitriles, such as acetonitrile and propionitrile, dimethyl sulfoxide, dimethylformamide, formamide, acetamide, dimethylacetamide, butyrolactone, 2-pyrrolidone and N-methylpyrrolidone.

The compounds of the general formula (I) are very generally suitable as solubilizers for oils, fats and waxes, as are described e.g. above as lipophilic carriers for cosmetic and pharmaceutical preparations. Reference is made here to this disclosure in its entirety. Also of suitability are the compounds of the general formula (I) as solubilizers for all of the aforementioned lipophilic cosmetic and pharmaceutical active ingredients and auxiliaries.

The compounds of the general formula (I) are also suitable as solubilizers for lipophilic cosmetic and/or pharmaceutical active ingredients. Suitable lipophilic substances are e.g. algae and plant extracts (e.g. Phlorogine from Biotechmarine, an algae extract for treating greasy skin, or e.g. Incromega V3 from Croda, a plant extract from Echiomega); anticellulite substances (e.g. CLA); antielastase and anticollagenase substances (e.g. unsaturated fatty acids, such as oleic acid or EPA); antiinflammatory substances (e.g. EPA=eicosapentaenoic acid); antioxidants (e.g. sage extract from Flavex, lipoic acid and derivatives thereof); ceramides (e.g. various ceramides from Cosmoferm); skin-calming and skin-smoothing active ingredients (e.g. bisabolol); moisturizers (e.g. glycerol monoisostearate, sucrose polysoyate); flavonoids (flavonoids include flavanols, flavanones, anthocyanidins, flavones and flavonols, such as e.g. sinensetin or polyphenols, such as those in green tea or grapes); phytosterols (such as 3-sitosterol from corn fiber oil); free-radical scavengers (e.g. ubiquinol derivatives such as coenzyme Q10); saponins (e.g. from Ginseng, liquorice root and horsechestnut); oxygen-binding substances (e.g. perfluorodecalin); sebum-reducing substances (e.g. 10-hydroxydecanoic acid); substances which promote circulation and therefore blood supply to the skin (e.g. nicotinic acid esters); terpenes (cosmetically and dermatologically relevant terpenes are listed in the Pharmazeutischene Zeitung, volume 22; 2006 by Sebastian Jager et al.); vitamins (retinol and derivatives, vitamin E and derivatives, such as tocotrienols or carotenes and carotenoids, such as lycopenes, lutein or fucoxanthine, vitamin D and derivatives).

In a further preferred embodiment, at least one compound (I) is used for the solubilization of at least one lipophilic organic UV filter substance. The UV filter substances are substances that are present in liquid or crystalline form at room temperature and which are able to absorb ultraviolet rays and release the absorbed energy again in the form of longer-wave radiation, e.g. heat. A distinction is made between UV-A filters and UV-B filters. The UV-A and UV-B filters can either be used individually or in mixtures.

Suitable lipophilic UV filter substances are:

Derivatives from the family of cinnamic acid, of dibenzoylmethane, of salicylic acid, of camphor, of triazines, of benzophenone, of diphenylacrylic acid, of benzotriazene, of benzylmalonic acid, of benzimidazole, of imidazolines, of p-aminobenzoic acid (PABA), of benzoxazoles, of 4,4-diarylbutadienecarboxylic acid, of dimers of alkylstyrenes and polymeric filters and silicone filters.

Typical lipophilic UV filters are:

3-Benzylidenecamphor or 3-benzylidenenorcamphor and derivatives thereof, e.g. 3-(4-methylbenzylidene)camphor, 3-(4′-trimethylammonium)benzylidenebornan-2-one methylsulfate (Mexoryl SO), 3,3′-(1,4-phenylenedimethine)bis(7,7-dimethyl-2-oxobicyclo-[2.2.1]heptane-1-methanesulfonic acid) and salts (Mexoryl SX), 3-(4′-sulfo)benzylidenebornan-2-ones and salts (Mexoryl SL), polymers of N-{(2 and 4)-[2-oxoborn-3-ylidene)methyl}benzyl]acrylamide (Mexoryl SW), 2-(2H-benzotriazol-2-yl)-4-methyl-6-(2-methyl-3-(1,3,3,3-tetramethyl-1-(trimethylsilyloxy)disiloxanyl)propyly phenol (Mexoryl SL), 4-aminobenzoic acid derivatives, preferably 2-ethylhexyl 4-(dimethylamino)benzoate, 2-octyl-4-(dimethylamino)benzoate and amyl-4-(dimethylamino)benzoate; esters of cinnamic acid, preferably 2-ethylhexyl-4-methoxycinnamate, propyl-4-methoxycinnamate, isoamyl 4-methoxycinnamate, 2-ethylhexyl 2-cyano-3,3-phenylcinnamate (octocrylene); esters of salicylic acid, preferably 2-ethylhexyl salicylate, 4-isopropylbenzyl salicylate, homomethyl salicylate; benzophenone derivatives, preferably 2-hydroxy-4-methoxybenzophenone, 2-hydroxy-4-methoxy-4′-methylbenzophenone, 2,2′-dihydroxy-4-methoxybenzophenone; esters of benzolmalonic acid, preferably di-2-ethylhexyl 4-methoxybenzmalonate; triazine derivatives, such as 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150) or bis(2-ethylhexyl) 4,4′-[(6-[4-((1,1-dimethylethyl)aminocarbonyl)phenylamino]-1,3,5-triazine-2,4-diyl)diimino]bisbenzoate (Uvasorb® HEB); 2,2-(methylenebis(6-(2H-benzotriazol-2-yl)-4-(1,1,3,3-tetramethylbutyl)phenol (Tinosorb M); 2,4-bis[4-(2-ethylhexyloxy)-2-hydroxyphenyl]-6-(4-methoxyphenyl)-1,3,5-triazine (Tinosorb S); propane-1,3-diones, e.g. 1-(4-tert-butylphenyl)-3-(4′-methoxyphenyl)propane-1,3-dione; ketotricyclo[5.2.1.0]decane derivatives and dimethicodiethylbenzolmalonate (Parsol SLX).

A further class of lipophilic compounds which can be solubilized with the help of at least one compound of the general formula (I) are dyes. Preferably, these are dyes suitable for cosmetic applications. These include e.g. cochineal red A (C.I. 16255), patent blue V (C.I. 42051), indigo tin (C.I. 73015), chlorophyllin (C.I. 75810), quinoline yellow (C.I. 47005), titanium dioxide (C.I. 77891), indanthrene blue RS (C.I. 69800).

The invention is described in more detail by reference to the following nonlimiting examples.

### EXAMPLES

Gas Chromatography (GC Analysis):

The preparation of the GC samples comprised a filtration of the sample amount, silylation with N-methyl-N-(trimethylsilyl)trifluoroacetamide (MSTFA) as solution in toluene (4:1) at 20° C., 1 hour. The analysis was carried out on an HP 6890 gas chromatograph.

### Preparation Examples

### Example 1

### Synthesis of 2,5-Di(Hydroxymethyl)Tetrahydrofuran Dilaurate

A 500 mL flask with mechanical stirrer, reflux condenser and dropping funnel was charged with a mixture of 2,5-di(hydroxymethyl)tetrahydrofuran (303 mmol, 40.0 g, 1.0 equiv.), dimethylaminopyridine (60.48 mmol, 7.39 g, 0.1 equiv.), diisopropylethylamine (605 mmol, 78.2 g, 2.0 equiv.) and THF (300 mL). Then, lauroyl chloride (605 mmol, 132 g, 2.0 equiv.) was added dropwise, during which the temperature did not exceed 35° C. Following complete addition, the reaction mixture was stirred for 2 hours at 25° C. The precipitated-out solid was filtered off and the filtrate was freed from solvent in vacuo. The resulting slightly yellowish oil was subjected to GC analysis. It comprised 2,5-di(hydroxymethyl)tetrahydrofuran dilaurate (93.6%), 2,5-di(hydroxymethyl)tetrahydrofuran monolaurate (2.8%), 2,5-di(hydroxymethyl)-tetrahydrofuran (0.1%) and lauric acid (2.1%).

¹H-NMR (500 MHz, CDCl₃) δ 4.06-3.98 (m, 2H), 3.91-3.78 (m, 1H), 2.17 (t, J=7.7 Hz, 2H), 1.85 (dd, J=8.2, 4.4 Hz, 1H), 1.57 (dd, J=7.8, 4.3 Hz, 1H), 1.47 (p, J=7.3 Hz, 2H), 1.23-0.92 (m, 18H), 0.72 (t, J=7.0 Hz, 3H) ppm

¹³C-NMR (126 MHz, CDCl₃) δ 173.2, 65.9, 33.8, 31.7, 29.4, 29.3, 29.1, 29.1, 28.9, 27.5, 24.7, 22.5, 22.3, 13.9 ppm

### Example 2

### Synthesis of 2,5-Di(Hydroxymethyl)Tetrahydrofuran Monolaurate

A 500 mL flask with mechanical stirrer, reflux condenser and dropping funnel was charged with a mixture of 2,5-di(hydroxymethyl)tetrahydrofuran (303 mmol, 40.0 g, 1.0 equiv.), dimethylaminopyridine (21.2 mmol, 2.59 g, 0.07 equiv.), diisopropylethylamine (605 mmol, 78.2 g, 2.0 equiv.) and THF (300 mL). Then, lauroyl chloride (212 mmol, 46.4 g, 0.7 equiv.) was added dropwise, during which the temperature did not exceed 35° C. Following complete addition, the reaction mixture was stirred for 2 hours at 25° C. The precipitated-out solid was filtered off and the filtrate was freed from solvent in vacuo. The residue was taken up in methyl tert-butyl ether (MTBE), washed with 10% strength aqueous NaOH and neutralized with dilute H₃PO₄. The organic phases were dried over Na₂SO₄ and the resulting colorless oil (106 g) was subjected to a GC analysis. It comprised 2,5-di(hydroxymethyl)tetrahydrofuran monolaurate (74.3%), 2,5-di(hydroxymethyl)tetrahydrofuran dilaurate (23.8%), 2,5-di(hydroxymethyl)tetrahydrofuran (0.5%) and lauric acid (0.7%).

### Example 3

### Synthesis of 2,5-Di(Hydroxymethyl)Tetrahydrofuran Dipalmitate

The preparation of 2,5-di(hydroxymethyl)tetrahydrofuran dipalmitate was carried out in accordance with the procedure described in example 1 from 2,5-di(hydroxymethyl)-tetrahydrofuran (303 mmol, 40.0 g, 1.0 equiv.), palmitoyl chloride (605 mmol, 166 g), dimethylaminopyridine (60.48 mmol, 7.40 g) and diisopropylethylamine (605 mmol, 78.2 g). The resulting product comprised 88.2% 2,5-di(hydroxymethyl)tetrahydrofuran dipalmitate and 5.1% 2,5-di(hydroxymethyl)tetrahydrofuran monopalmitate. The mixture was produced as a colorless, wax-like solid, melting point: 50.5° C.

¹H-NMR (500 MHz, CDCl₃) δ 4.20-4.12 (m, 4H), 4.02-3.95 (m, 2H), 2.35-2.28 (m, 4H), 1.98 (tdd, J=6.7, 4.8, 2.6 Hz, 2H), 1.69 (dtd, J=10.8, 6.6, 2.3 Hz, 2H), 1.59 (p, J=7.3 Hz, 4H), 1.22 (s, 41H), 0.85 (t, J=7.0 Hz, 6H) ppm.

¹³C-NMR (126 MHz, CDCl₃) δ 173.8, 66.2, 34.2, 32.0, 29.7, 29.7, 29.7, 29.7, 29.7, 29.6, 29.5, 29.4, 29.3, 29.2, 27.7, 24.9, 22.7, 14.2 ppm.

### Example 4

### ENZYMATIC Synthesis of 2,5-Di(Hydroxymethyl)Tetrahydrofuran Distearate (a) and 2,5-Di(Hydroxymethyl)Tetrahydrofuran Palmitate (b)

The respective acid component was heated to 75° C. with 2,5-di(hydroxymethyl)-tetrahydrofuran in a 1 L stirred reactor and mixed. The esterification reaction was started by adding 1% by weight of commercially available Novozym® 435 (Lipase B from Candida antarctica immobilized on a polymethacrylate support). Removal of the resulting water of reaction was carried out by a combination of reduced pressure (50 to 10 mbar) and gassing with nitrogen as stripping gas (0 to 11 L/h*kg (STP)). The course of the reaction was monitored by means of acid number. At the end of the reactions, the catalyst was separated off from the product by means of filtration.

Initial weights of the raw materials:
a) 134 g of 2,5-di(hydroxymethyl)tetrahydrofuran,
   568 g of stearic acid
b) 83 g of 2,5-di(hydroxymethyl)tetrahydrofuran,
   317 g of palmitic acid

Conversion Progress Examples 4 a) and 4 b):

| THF glycol distearate (a) | | THF glycol dipalmitate (b) | |
|---|---|---|---|
| Time [h] | Acid conversion [%] | Time [h] | Acid conversion [%] |
| 0 | 0.0 | 0 | 0.0 |
| 2 | 74.5 | 2 | 71.8 |
| 7 | 96.3 | 6 | 94.9 |
| 30 | 99.4 | 27 | 99.3 |

### Example 5

### Enzymatic Synthesis of a 2,5-Di(Hydroxymethyl)Tetrahydrofuran Diester of a Fatty Acid Mixture of Behenic Acid and Stearic Acid

230 g of behenic acid (technical-grade: behenic acid 90%, arachic acid 8%, stearic acid, lignoceric acid in each case <2%) and 100 g of stearic acid (stearic acid >98%; palmitic acid, behenic acid in each case <2%) were heated to 85° C. with 70 g of 2,5-di(hydroxymethyl)tetrahydrofuran in a 1 L stirred reactor and mixed. The esterification reaction was started by adding 1% by weight of commercially available Novozym 435. The removal of the resulting water of reaction was carried out by the combination of reduced pressure (50 to 10 mbar) and gassing with nitrogen as stripping gas (0 to 11 L/h*kg (STP)). The progress of the reaction was monitored by means of acid number. At the end of the reactions, the catalyst was separated off from the product by means of filtration.

Conversion Progress Example 5:

| Time [h] | Acid conversion [%] |
|---|---|
| 0 | 0 |
| 2 | 83.1 |
| 6 | 98.7 |
| 23 | 99.9 |

### Example 6

### Enzymatic Synthesis of a of 2,5-Di(Hydroxymethyl)Tetrahydrofuran Diester of a Technical-Grade Fatty Acid Mixture Based on Hydroxystearic Acid

334 g of a technical-grade fatty acid mixture (Edenor® OSSG, Henkel), which comprises about 85% 12-hydroxystearic acid, 10% stearic acid and, as remainder, primarily C₁₆-, C₂₀- and C₂₂-fatty acids (in each case <2%), were heated to 85° C. with 67 g of 2,5-di(hydroxymethyl)tetrahydrofuran (0.5076 mol) in a 1 L stirred reactor and mixed. The esterification reaction was started by adding 1% by weight (4 g) of commercially available enzyme Novozym 435. The removal of the resulting water of reaction was carried out by means of the combination of vacuum (50 to 10 mbar) and gassing with nitrogen as entrainer gas (0 to 11 L/h*kg (STP)). The progress of the reaction was monitored by means of acid number. At the end of the reactions, the catalyst was separated off from the product by means of filtration.

Conversion Progress:

| Time [h] | Acid conversion [%] |
|---|---|
| 0 | 0.0 |
| 2 | 67.2 |
| 6 | 91.5 |
| 23 | 99.7 |

1. A compound of the general formula (I) 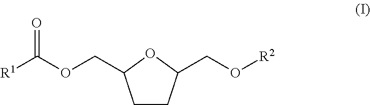
in which
R² is C(═O)R^{1′} and where R¹ and R^{1′}, independently of one another, are unbranched or branched C₅-C₃₅-alkyl or unbranched or branched C₅-C₃₅-alkenyl with 1, 2, 3 or more than 3 double bonds, where C₅-C₃₅-alkyl and C₅-C₃₅-alkenyl can in each case be substituted by at least one hydroxyl group,
or
R² is hydrogen and R¹ is unbranched or branched C₅-C₃₅-alkyl or unbranched or branched C₅-C₃₅-alkenyl with 1, 2, 3 or more than 3 double bonds, where C₅-C₃₅-alkyl and C₅-C₃₅-alkenyl can be substituted by at least one hydroxyl group and/or can have at least one epoxy group.

2. The compound of the general formula (I) according to claim 1, wherein at least one of the radicals R¹ and R^{1′} is unbranched or branched C₁₁-C₂₁-alkyl or unbranched or branched C₁₁-C₂₁-alkenyl with 1, 2 or 3 double bonds, where C₁₁-C₂₁-alkyl and C₁₁-C₂₁-alkenyl can be substituted by at least one hydroxyl group.

3. The compound of the general formula (I) according to claim 1, wherein R² is C(═O)R^{1′} and the radicals R¹ and R^{1′} have the same meaning.

4. The compound of the general formula (I) according to claim 1, wherein R² is hydrogen.

5. A process for the preparation of compounds of the general formula (I), 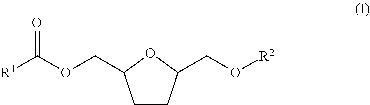
in which R¹ and R² are as defined in any one of claims 1 to 4, in which 2,5-di(hydroxymethyl)tetrahydrofuran is reacted with at least one compound R¹—COOH and, if R² is C(═O)R^{1′}, and R¹ and R^{1′} have a different meaning, additionally with a compound R^{1′}—COOH different therefrom in the presence of an enzymatic esterification catalyst.

6. The process according to claim 5, where the enzymatic esterification catalyst comprises at least one enzyme which is selected from hydrolases.

7. A cosmetic or pharmaceutical composition comprising at least one compound of the general formula (I) as defined in claim 1 and at least one cosmetic or pharmaceutical active ingredient and/or auxiliary different therefrom.

8. The cosmetic composition according to claim 7 in the form of a cream, mousse, milk, lotion, mascara, stage makeup, soap of liquid to solid consistency, a foam, gel, spray, stick, washing, showering or bathing preparation of liquid to gel-like consistency, eyeshadow, eyeliner, blusher, powder or strip.

9. A detergent, cleaner or dishwashing composition or rinse aid, comprising at least one compound of the general formula (I), as defined in claim 1, and at least one surfactant different therefrom.

10. The use of at least one compound of the general formula (I), as defined in claim 1, as an interface-active compound.

11. The use according to claim 17 as a surfactant, where R² is hydrogen.

12. The use according to claim 17 as a solubilizer, wherein
R² is C(═O)R1′ and wherein at least one of the radicals R¹ and R^{1′} is an unbranched or branched C₁₁-C₃₅-alkyl or unbranched or branched C₁₁-C₃₅-alkenyl with 1, 2, 3 or more than 3 double bonds, wherein C₁₁-C₃₅-alkyl and C₁₁-C₃₅-alkenyl can in each case be substituted by at least one hydroxyl group,
or
R² is hydrogen and R¹ is unbranched or branched C₁₁-C₃₅-alkyl or unbranched or branched C₁₁-C₃₅-alkenyl with 1, 2, 3 or more than 3 double bonds, where C₁₁-C₃₅-alkyl and C₁₁-C₃₅-alkenyl can be substituted by at least one hydroxyl group and/or can have at least one epoxy group.

13. The use of at least one compound of the general formula (I), as defined in claim 1, as rheology modifiers, wherein
R² is hydrogen and R¹ is unbranched or branched C₅-C₃₅-alkenyl with 1, 2, 3 or more than 3 double bonds, where C₅-C₃₅-alkenyl can be substituted by at least one hydroxyl group and/or can have at least one epoxy group.

14. The use of at least one compound of the general formula (I), as defined in claim 1, as emollients, wherein
R² is C(═O)R^{1′} and where R¹ and R^{1′}, independently of one another, are unbranched or branched C₅-C₂₃-alkyl or unbranched or branched C₅-C₂₃-alkenyl with 1, 2, 3 or more than 3 double bonds.

15. The use of at least one compound of general formula (I) as defined in claim 1
in cosmetic compositions,
in pharmaceutical compositions,
in detergents and cleaners,
in dishwashing compositions and in rinse aids,
in hygiene products,
in crop protection compositions,
in paints, coating compositions, adhesives, leather-treatment or textile-treatment compositions,
in the development and/or exploitation of subterranean natural oil and/or natural gas deposits.

16. A process for preparing compounds of the general formula (I) 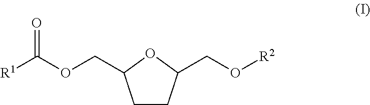
in which R¹ and R² are as defined in claim 1, in which 2,5-di(hydroxymethyl)tetrahydrofuran is reacted with at least one acid halide R¹—C(═O)X and, if R² is C(═O)R^{1′} and R¹ and R^{1′} have a different meaning, additionally with at least one acid halide R^{1′}—C(═O)X, where X is Br or Cl, in the presence of at least one tertiary amine.

17. The use according to claim 10 wherein the interface-active compound is a surfactant, emulsifier, solubilizer, or foam former.

18. The method of claim 6 wherein the hydrolases comprises lipases and esterases.

19. The method of claim 18 wherein the lipase comprises at least one of Candida rugose, Candida antiartica, Thermomyces lanunginosa, and Rhizomucor miehei.

20. The method of claim 18 wherein the esterase is from pig liver.
